(19) United States
(12) Patent Application Publication
(10) Pub. No.: US 2023/0302065 A1
(43) Pub. Date: Sep. 28, 2023

(54) COMPOSITION WITH LACTOBACILLUS PARACASEI AND A METHOD TO TREAT NASOPHARYNGEAL CARCINOMA THROUGH PYROPTOSIS OR CELL CYCLE ARREST

(71) Applicant: GenMont Biotech Incorporation, Tainan City (TW)

(72) Inventors: Wan-Hua TSAI, Kaohsiung City (TW); I-Ling HSU, Tainan City (TW); Wen-Wei CHANG, Taichung City (TW); Chin-Chun LIN, New Taipei City (TW)

(21) Appl. No.: 17/862,766

(22) Filed: Jul. 12, 2022

(30) Foreign Application Priority Data
Mar. 24, 2022 (TW) 111111049

(51) Int. Cl. A61K 35/747; A23L 33/135; A61P 35/00; C12N 1/20

(52) U.S. Cl. CPC A61K 35/747; A23L 33/135; A61P 35/00; C12N 1/205; C12R 2001/225

(57) ABSTRACT

A composition with Lactobacillus paracasei and a method to treat nasopharyngeal carcinoma through induction of pyroptosis or cell cycle arrest; Lactobacillus paracasei GMNL-653 or heat-killed whole-bacterial liquids thereof are taken as effective ingredients for inhibiting proliferations of nasopharyngeal carcinoma cells or reducing a probability of nasopharyngeal carcinoma development through pyroptosis or cell cycle arrest instead of the mechanism of apoptosis.

### BACKGROUND OF THE INVENTION

### Field of the Invention

The present invention relates to a technique which relies on Lactobacillus paracasei to resist nasopharyngeal carcinoma, particularly a technique to inhibit nasopharyngeal carcinoma through pyroptosis or cell cycle arrest.

### Description of the Prior Art

Nasopharyngeal carcinoma is believed to be a cancer with regional differences for found cases in which many patients live in Southeast Asia or South China particularly and are associated with diets, genetic characteristics, exposures to some substances and highly correlated with Epstein-Barr virus infection. Most nasopharyngeal carcinoma patients undergo radiotherapy, which is criticized for significant early complications and insufferable pains, or as the adjuvant therapy for cervical lymph nodes removed surgically but still existing under radiant exposures. The radiotherapy interferes with salivary secretion such that a patient whose saliva gradually dries up suffers from permanent sequelae like pharyngitis sicca and middle ear effusion or fibrosis of the skull base, oral mucositis, etc. Some researchers argued that a patient diagnosed with nasopharyngeal carcinoma may risk thyroid hypofunction after the radiotherapy. Accordingly, it is necessary to find a low-risk therapy for nasopharyngeal carcinoma.

Defined as live but nonpathogenic microorganisms, probiotics with the bacterial count controlled appropriately is beneficial to a host and well known for functions like improving intestinal bacterial flora, reducing hazards of pathogenic bacteria to a human being, strengthening immunoreactions, abating allergic reactions and serving as nutritional supplements of a cancer survivor. Moreover, probiotics are critically acclaimed for regulating inflammatory responses, reinforcing epithelial barriers and preventing chronic inflammations of intestines. With probiotics and prebiotics integrated, the diet modification for the colorectal cancer is considered as a good remedy to circumvent carcinogens, alleviate carcinogenicity of a carcinogen and prevent tumor development.

In addition to live probiotics or products thereof, bacteria-free supernatants of heat-treated probiotic liquids and purified critical ingredients are also beneficial. Compared with live probiotics, heat-inactivated probiotics or purified critical ingredients thereof have the advantages of safe manipulation. Comparatively, the heat-inactivated probiotics formulation administered to a cancer patient is a safe choice when metastasis of cancer cells to the patient's bone marrows, chemotherapy or radiotherapy gives rise to a compromised immune system. Heat-killed probiotic L. brevis promotes expressions of pro-apoptotic proteins BAX, Caspase-3 and Caspase-9 mRNA but abates the expression of anti-apoptosis protein Bcl2 in HT-29 cells. It was discovered that culture supernatants of heat-killed Bacillus coagulans Unique IS2 strain increases expressions of the pro-apoptotic protein BAX but reduces the expression of the anti-apoptosis protein Bcl2, resulting in the elevation of caspase-3 activity, an increase of released cytochrome C and inhibition of the colorectal cancer cell proliferation. The test results for the anti-tumor effect in BALB/c mice with the normal immunologic function suggest oral administration of heat-killed L. plantarum BF-LP284 is better than live L. plantarum BF-LP284. As disclosed in researches, heat-killed L. plantarum BF-LP284 stimulates INF-γ production in spleen cells, and triggers the migration of T cells with the antitumor activity from the spleen to a tumor to inhibit tumor growth. Many researchers have demonstrated the function of heat-killed probiotics to inhibit proliferations of cancer cells and tumor development. L. paracasei K5 adheres to surfaces of colorectal cancer cells and inhibits proliferations of cancer cells by triggering apoptosis. L. paracasei SR4 kills cervical cancer cells through apoptosis and increasing cellular oxidative stress.

However, apoptosis is the dominant strategy adopted by most existing probiotics-based techniques to annihilate cancer cells instead of pyroptosis or cell cycle arrest and there is no evidence that nasopharyngeal carcinoma cells can be killed by probiotics.

### SUMMARY OF THE INVENTION

Against the above background, the patent applicant deeply comprehended the shortcomings and drawbacks in the prior art, and devoted to an innovative design and successfully extracted probiotics, Lactobacillus paracasei, resisting nasopharyngeal carcinoma, inhibiting proliferations of nasopharyngeal carcinoma through pyroptosis or cell cycle arrest, and verified as a new and safe strategy for treatment of nasopharyngeal carcinoma after years of research.

To this end, the patent applicant provides a composition in the present disclosure comprising Lactobacillus paracasei GMNL-653 (deposited under the number of BCRC 910721 or CCTCC M 2016226) or heat-killed whole-bacterial liquids thereof as effective ingredients to inhibit proliferations of nasopharyngeal carcinoma cells or reduce a probability of nasopharyngeal carcinoma development through pyroptosis or cell cycle arrest.

In one embodiment of the present disclosure, proliferations of nasopharyngeal carcinoma cells inhibited or a probability of nasopharyngeal carcinoma development reduced is not induced by the effective ingredients through apoptosis.

In one embodiment of the present disclosure, the effective ingredients comprise Lactobacillus paracasei GMNL-653 or peptidoglycan and lipoteichoic acid in heat-killed whole-bacterial liquids of Lactobacillus paracasei GMNL-653.

In one embodiment of the present disclosure, the effective ingredients comprise the portion of separated heat-killed microbial supernatants containing molecules with the molecular weight greater than 3 kDa in Lactobacillus paracasei GMNL-653.

In one embodiment of the present disclosure, the composition is manufactured as a pharmaceutical composition, nutritional supplements or health food.

In one embodiment of the present disclosure, the composition further comprises a pharmaceutically accepted vehicle.

In one embodiment of the present disclosure, the composition is manufactured as a solution, a suspension liquid, an emulsion, powders, a pastille, a pill, syrup, an oral ingot, a tablet, a chewing gum, a stiff paste or a capsule.

In one embodiment of the present disclosure, the composition further comprises an edible material including, without limitation, water, liquid dairy product, milk, concentrated milk, yogurt, frozen yogurt, Lactobacillus fermented beverage, powdered milk, ice cream, cheese, dry cheese, soymilk, fermented soymilk, vegetable juice, fruit juice, sports drink, dessert, jelly, candy, baby food, health food, animal food, Chinese medicinal herb or dietary supplement.

Furthermore, the patent applicant provides a method to treat nasopharyngeal carcinoma with Lactobacillus paracasei GMNL-653; the method is aimed at administration of Lactobacillus paracasei GMNL-653 (deposited under the number BCRC 910721 or CCTCC M 2016226) or heat-killed whole-bacterial liquids thereof as effective ingredients to an individual for inhibiting proliferations of nasopharyngeal carcinoma or reducing a probability of nasopharyngeal carcinoma development through pyroptosis or cell cycle arrest.

In one embodiment of the present disclosure, proliferations of nasopharyngeal carcinoma cells inhibited or a probability of nasopharyngeal carcinoma development reduced is not induced by effective ingredients through apoptosis.

In one embodiment of the present disclosure, the effective ingredients comprise Lactobacillus paracasei GMNL-653 or peptidoglycan and lipoteichoic acid in heat-killed whole-bacterial liquids of Lactobacillus paracasei GMNL-653.

In one embodiment of the present disclosure, the effective ingredients comprise the portion of separated heat-killed microbial supernatants containing molecules with the molecular weight greater than 3 kDa in Lactobacillus paracasei GMNL-653.

In one embodiment of the present disclosure, proliferations of nasopharyngeal carcinoma cells are inhibited by the effective ingredients through generation and accumulation of reactive oxygen species in nasopharyngeal carcinoma cells.

In one embodiment of the present disclosure, a probability of nasopharyngeal carcinoma development is reduced by the effective ingredients through inhibition of self-renewals of nasopharyngeal carcinoma stem cells.

In one embodiment of the present disclosure, the effective dosage of peptidoglycan or lipoteichoic acid in Lactobacillus paracasei GMNL-653 or heat-killed whole-bacterial liquids thereof ranges from 10 μg/ml to 50 μg/ml.

In one embodiment of the present disclosure, the effective ingredients are administered to a patient through each of oral administration, injection, application, spray and a patch.

In the present disclosure, the patent applicant provides Lactobacillus paracasei GMNL-653 which inhibits proliferations of nasopharyngeal carcinoma cells or reduces a probability of nasopharyngeal carcinoma development through pyroptosis or cell cycle arrest, generation or accumulation of reactive oxygen species in nasopharyngeal carcinoma cells, or inhibition of self-renewals of nasopharyngeal carcinoma stein cells instead of the mechanism of apoptosis. Moreover, the patent applicant provides a composition with Lactobacillus paracasei GMNL-653 as effective ingredients which inhibits nasopharyngeal carcinoma through pyroptosis or cell cycle arrest and features low side effects due to the composition based on probiotics as effective ingredients.

### BRIEF DESCRIPTION OF THE DRAWINGS

The techniques of present invention would be more understandable from the detailed description given herein below and the accompanying figures are provided for better illustration, and thus description and figures are not limitative for present invention, and wherein:

FIG. 1 illustrates the effect of heat-killed whole-bacterial liquids (HK-653) of Lactobacillus paracasei GMNL-653 on proliferations of nasopharyngeal carcinoma cells according to the MTT assay for survivals of nasopharyngeal carcinoma cells, NPC-BM1, (A of FIG. 1) and nasopharyngeal carcinoma cells, NPC-076, (B of FIG. 1) in one embodiment.

FIG. 2 illustrates the effect of heat-killed whole-bacterial liquids (HK-653) of Lactobacillus paracasei GMNL-653 on proliferations of nasopharyngeal carcinoma cells according to the clonogenic assay in one embodiment wherein appearances of nasopharyngeal carcinoma cells cultivated in culture dishes are shown in A of FIG. 2 and quantitative results of nasopharyngeal carcinoma cells, NPC-BM1, and nasopharyngeal carcinoma cells, NPC-076, are shown in B of FIG. 2 and C of FIG. 2, respectively.

FIG. 3 illustrates the effect of heat-killed whole-bacterial liquids (HK-653) of Lactobacillus paracasei GMNL-653 on the cell cycle of nasopharyngeal carcinoma cells, NPC-BM1, (A of FIG. 3) and nasopharyngeal carcinoma cells, NPC-076, (B of FIG. 3) in one embodiment.

FIG. 4 illustrates the effect of heat-killed whole-bacterial liquids (HK-653) of Lactobacillus paracasei GMNL-653 on expressions of proteins correlated with the cell cycle of nasopharyngeal carcinoma cells in one embodiment.

FIG. 5 illustrates the effect of heat-killed whole-bacterial liquids (HK-653) of Lactobacillus paracasei GMNL-653 on apoptosis of nasopharyngeal carcinoma cells, NPC-BM1, (A of FIG. 5) and nasopharyngeal carcinoma cells, NPC-076, (B of FIG. 5) in one embodiment.

FIG. 6 illustrates the effect of heat-killed whole-bacterial liquids (HK-653) of Lactobacillus paracasei GMNL-653 on the mitochondrial membrane potential of nasopharyngeal carcinoma cells in one embodiment.

FIG. 7 illustrates the effect of ROS (reactive oxygen species) inhibitors as well as heat-killed whole-bacterial liquids (HK-653) of Lactobacillus paracasei GMNL-653 on proliferations of nasopharyngeal carcinoma cells, NPC-BM1, (A of FIG. 7) and nasopharyngeal carcinoma cells, NPC-076, (B of FIG. 7) in one embodiment.

FIG. 8 illustrates the effect of heat-killed whole-bacterial liquids (HK-653) of Lactobacillus paracasei GMNL-653 on accumulation of ROS in nasopharyngeal carcinoma cells, NPC-BM1, (A of FIG. 8) and nasopharyngeal carcinoma cells, NPC-076, (B of FIG. 8) in one embodiment.

FIG. 9 illustrates the effect of heat-killed whole-bacterial liquids (HK-653) of Lactobacillus paracasei GMNL-653 on proteins related to DNA damages and DNA repair pathways in nasopharyngeal carcinoma cells in one embodiment.

FIG. 10 illustrates the effect of heat-killed whole-bacterial liquids (HK-653) of Lactobacillus paracasei GMNL-653 on pyroptosis-related proteins in nasopharyngeal carcinoma cells in one embodiment.

FIG. 11 illustrates the effect of heat-killed whole-bacterial liquids (HK-653) of Lactobacillus paracasei GMNL-653 on self-renewal activity of nasopharyngeal carcinoma stein cells in one embodiment wherein appearances of tumor spheroids formed from nasopharyngeal carcinoma cells are shown in A of FIG. 11 and quantitative test results for tumor spheroids formed from nasopharyngeal carcinoma cells, NPC-BM1, and nasopharyngeal carcinoma cells, NPC-076, are shown in B of FIG. 11.

FIG. 12 illustrates the effect of heat-killed whole-bacterial liquids (HK-653) of Lactobacillus paracasei GMNL-653 on proteins related to the SHH pathway (for self-renewals of nasopharyngeal carcinoma stein cells) in one embodiment.

FIG. 13 illustrates the effect of heat-killed whole-bacterial liquids (HK-653), heat-killed microbial supernatants (HK-653-s) or heat-killed bacterial pellets (HK-653-p) of Lactobacillus paracasei GMNL-653 on proliferations of nasopharyngeal carcinoma cells, NPC-BM1, (A of FIG. 13) and nasopharyngeal carcinoma cells, NPC-076, (B of FIG. 13) in one embodiment.

FIG. 14 illustrates the effect of heat-killed whole-bacterial liquids (HK-653), heat-killed microbial supernatants (HK-653-s) or heat-killed bacterial pellets (HK-653-p) of Lactobacillus paracasei GMNL-653 on proteins related to the cell cycle of nasopharyngeal carcinoma cells in one embodiment.

FIG. 15 illustrates the effect of two portions of heat-killed microbial supernatants of Lactobacillus paracasei GMNL-653 containing molecules with the molecular weight greater than 3 kDa (“sup>3K”) with the molecular weight less than 3 kDa (“sup<3K”) on proliferations of nasopharyngeal carcinoma cells, NPC-BM1, (A of FIG. 15) and nasopharyngeal carcinoma cells, NPC-076, (B of FIG. 15) in one embodiment.

FIG. 16 illustrates the effect of lipoteichoic acid or peptidoglycan in heat-killed bacterial pellets of Lactobacillus paracasei GMNL-653 on proliferations of nasopharyngeal carcinoma cells, NPC-BM1, (A of FIG. 16) and nasopharyngeal carcinoma cells, NPC-076, (B of FIG. 16) in one embodiment.

### DETAILED DESCRIPTION OF THE INVENTION

### Terminology Definition

The technical and scientific terminologies common in the art of biotech are widely adopted in the patent specification; the definitions of these technical and scientific terminologies are given for clear and consistent understanding of the patent specification as well as claims hereinafter. Other terminologies not defined particularly hereinafter are well known to and commonly understood by persons skilled in the art.

A singular noun joined by “a/an”, “one” or “the” in the patent specification or claims may refer to more than one object unless otherwise specified.

The word like “or” or “and” refers to “and/or” unless otherwise specified. Moreover, the word like “comprise” or “include” is an open-ended term. The descriptions in a previous section refer to general involvement but are not interpreted as restrictions to the subject of the present invention.

The terminology of “nasopharyngeal carcinoma” adopted herein is a general term for epithelial cell carcinoma originating from nasopharynx and covered by mucosa and includes, without limitation, squamous cell carcinoma, non-keratinizing carcinoma, undifferentiated carcinoma, ascending type of nasopharyngeal carcinoma, descending type of nasopharyngeal carcinoma, mixed type of nasopharyngeal carcinoma, ulcerative type of nasopharyngeal carcinoma, lobular type of nasopharyngeal carcinoma, protruded type of nasopharyngeal carcinoma, protruded/lobular type nasopharyngeal carcinoma, infiltrating type of nasopharyngeal carcinoma, bulging type of nasopharyngeal carcinoma and granular type of nasopharyngeal carcinoma. In the present disclosure, nasopharyngeal carcinoma cells to be inspected, analyzed, classified or treated comprise precancerous (i.e., benign), malignant, metastatic, pre-metastatic or non-metastatic cells.

The terminology of “treatment”, “for the treatment of” or the like adopted herein means administration of a medicine for a certain effect on a patient. The effect denotes a certain disease and/or symptoms thereof partially or completely cured through treatment. As shown in the present disclosure, the “treatment” means any cure of nasopharyngeal carcinoma diagnosed in a mammal (particularly a human being) and including (a) control of a disease, that is, progression of a disease inhibited and (b) a disease in remission, that is, symptoms relieved. For treatment of a tumor (for example, nasopharyngeal carcinoma), proliferation and metastasis of a tumor is alleviated by a therapeutic agent directly.

The terminology of “cell culture” or the like adopted herein means cells alive in ex vivo. The terminology of “cell culture”, however, should be interpreted as a general term for culture of a single cell, a tissue or an organ.

The terminology of “tumor” adopted herein means growths and proliferations of all neoplastic (benign or malignant), precancerous or cancerous cells and tissues.

The terminology of “pharmaceutically acceptable” means a substance or a composition and other components of a pharmaceutical concoction thereof being compatible with each other but not aggravating a patient's symptoms.

The effective ingredients or the composition in the present disclosure as well as at least a pharmaceutically acceptable vehicle can be chosen by a person with common and well-known knowledge in the art for preparation of a formulation applicable to the composition. The formulation includes, without limitation, a solution, an emulsion, a suspension liquid, powders, a pastille, an oral ingot, a tablet, a chewing gum, a capsule and another similar formulation applicable to the present invention.

The terminology of “pharmaceutically acceptable vehicle” comprises one or more types of ingredients selected from: a solvent, an emulsifier, a suspension agent, a decomposing agent, a binding agent, an excipient, a stabilizing agent, a chelating agent, a diluent, a gelling agent, a preservative, a lubricating agent, a surfactant and another similar vehicle applicable to the present invention.

One or more co-solvents, buffer agents, coloring agents and flavoring agents common in the pharmaceutical industry can be discretionarily added in the composition as required.

The terminology of “pharmaceutically acceptable excipient” includes, without limitation, at least one ingredient selected from a polymer, a resin, a plasticizer, a padding, a lubricating agent, a diluent, an adhesive, a disintegrant, a solvent, a co-solvent, an interfacial agent, a preservative, a sweetening agent, a flavoring agent, a pharmaceutical-grade dye or pigment and a binding agent.

The terminology of “pharmaceutical composition” means a solid or liquid composition with a form, concentration and purity applicable to medicine administration for a patient from whom an expected physiological change is induced after administration; the pharmaceutical composition is sterile and/or non-pyrogenic.

The terminology of “effective dosage” means a certain dosage for expected biological feedback from a creature but not recovery of the creature. As comprehended by a person with common knowledge in the art, the effective dosage of the pharmaceutical composition may change with following factors like an expected biological endpoint, a bio-activator to be delivered, composition of an encapsulating matrix and a target tissue.

The terminology of “related to”, “correlated with” or the like adopted herein means two events or cases with a statistical correlation between each other, for example, two numbers, data groups and the like. In the case of two numbers, for example, the positive correlation denotes a number increasing with another rising number and the negative correlation denotes a number decreasing with another rising number.

[Lactobacillus paracasei]

In the present disclosure, Lactobacillus paracasei, which inhibits proliferations of nasopharyngeal carcinoma through pyroptosis or cell cycle arrest, is not limited to a specific bacterial strain and shown in an embodiment for Lactobacillus paracasei under the number of BCRC 910520 as well as Lactobacillus paracasei GMNL-653 under the number of BCRC 910721 deposited in the Food Industry Research and Development Institute, Taiwan, and under the number of CCTCC M 2016226 deposited in the China Center for Type Culture Collection (CCTCC); Lactobacillus paracasei can be one type of bacterial strain or two or more types of bacterial strains. The source of Lactobacillus paracasei is not specially designated in the present disclosure and commercially available or isolated from a lab or clinically.

[Method to Treat Nasopharyngeal Carcinoma]

The present invention discloses a method to treat nasopharyngeal carcinoma with which either a therapeutically effective dosage of Lactobacillus paracasei or heat-killed whole-bacterial liquids thereof, as effective ingredients, are administered to a patient for preventing or treating nasopharyngeal carcinoma through pyroptosis or cell cycle arrest; the effect ingredients in heat-killed whole-bacterial liquids comprise heat-killed microbial supernatants and heat-killed bacterial pellets (including lipoteichoic acid and peptidoglycan).

The method to treat nasopharyngeal carcinoma disclosed herein is applicable to an individual mammal (human being particularly) which has come down with or risked a tumor as shown in the present disclosure.

The method to treat nasopharyngeal carcinoma disclosed herein is to administer Lactobacillus paracasei or heat-killed whole-bacterial liquids thereof to an individual (for example, a human being diagnosed with nasopharyngeal carcinoma) for inhibition of nasopharyngeal carcinoma cells.

The method is also applicable to inhibiting proliferation of a patient's tumor in size, alleviating the disease burden of a tumor patient and/or improving clinical outcomes.

[Administration of Lactobacillus paracasei]

The administration of Lactobacillus paracasei or heat-killed whole-bacterial liquids thereof to nasopharyngeal carcinoma tissues as the target is effectuated through different pathways including intratumoral administration, intravenous injection, intracutaneous injection, subcutaneous injection, oral administration (for example, inhalation), percutaneous injection (i.e., topical injection), mucosal administration, intraperitoneal administration, intra-artery administration or rectal administration. Other applicable pathways for administration of Lactobacillus paracasei or heat-killed whole-bacterial liquids thereof are effectuated through oral administration, transnasal administration, nasopharyngeal administration, parenteral administration, intestinal administration, gastric tube administration, topical injection, percutaneous injection, subcutaneous injection, intramuscular injection, pastille, solid, pulvis, liquid, aerosol, intralesional injection into a tumor, intralesional injection around a tumor, intravenous injection or arterial injection. Moreover, the topical or systemic administration in which excipients are added or not added is available; medications are administered into or around an individual's tumor by a slow release mode. Among all pathways for administration, it is preferred that Lactobacillus paracasei or heat-killed whole-bacterial liquids thereof are administered to a patient through each of oral administration, injection, application, spray or a patch.

[Dosage]

The method to treat nasopharyngeal carcinoma disclosed herein is to administer a therapeutically effective dosage of Lactobacillus paracasei or heat-killed whole-bacterial liquids thereof to an individual as required. In detail, the administration depends on lots of factors: purpose of medicine administration; healthy conditions, age and category (for example, human being, non-human primate and primate) of an individual to be treated; formulation of Lactobacillus paracasei or heat-killed whole-bacterial liquids thereof; medical conditions evaluated by a clinician; others. It is expected that a routine test is conducted through administration of Lactobacillus paracasei or heat-killed whole-bacterial liquids thereof with a therapeutically effective dosage of relatively wide tolerance. For maximum deaths of nasopharyngeal carcinoma cells, for example, the therapeutically effective dosage of Lactobacillus paracasei or heat-killed whole-bacterial liquids thereof ranges from 1×10⁸ cells/ml to 1×10⁹ cells/ml.

Furthermore, the therapeutically effective dosage of peptidoglycan in Lactobacillus paracasei or heat-killed whole-bacterial liquids thereof ranges from 10 μg/ml to 50 μg/ml; the therapeutically effective dosage of lipoteichoic acid in Lactobacillus paracasei or heat-killed whole-bacterial liquids thereof ranges from 10 μg/ml to 50 μg/ml.

The present invention is elaborated in following embodiments which are interpreted as examples disclosed hereinafter only but not regarded as evidences to restrict the range of the present invention in any way. Moreover, Lactobacillus paracasei and materials hereinafter are commercially available.

[Cultivation of Lactobacillus paracasei and Preparation of Heat-Killed Whole-Bacterial Liquids]

Lactobacillus paracasei GMNL-653 and Lactobacillus paracasei under the number of BCRC 910520 are cultivated in MRS (DeMan-Rogosa-Sharpe) broths in an incubator (37° C.; 5% CO₂) for 24 hours and further sub-cultured in MRS broths for another 24 hours. With bacterial cultures centrifuged (4000 rpm; 15 min), collected bacterial pellets are rinsed twice with sterile water and re-dissolved in sterile water for estimation of the bacterial count at OD 590 nm and the bacterial count adjusted as 1×10¹⁰ (cells/ml). After 15-minute heating at 121° C., heat-killed whole-bacterial liquids of Lactobacillus paracasei are prepared. For that matter, the heat-killed whole-bacterial liquids of Lactobacillus paracasei GMNL-653 are hereinafter referred to as HK-653.

[Separations and Preparations of Heat-Killed Microbial Supernatants, Heat-Killed Bacterial Pellets, Lipoteichoic Acid and Peptidoglycan of Lactobacillus paracasei]

To recognize whether the active ingredients are in heat-killed microbial supernatants or heat-killing bacterial pellets, the heat-killed whole-bacterial liquids (1×10¹⁰ cells/ml) of Lactobacillus paracasei GMNL-653 are centrifuged under 12000 g for 10 minutes and then heat-killed microbial supernatants are collected and filtered through 0.22 μm filters for complete removals of bacterial pellets. The filtered heat-killed microbial supernatants of Lactobacillus paracasei GMNL-653 (HK-653-s, sup) are divided into one portion containing molecules with the molecular weight greater than 3 kDa (Sup>3K) and another portion containing molecules with the molecular weight less than 3 kDa (Sup<3K) using a centrifugal filter with a membrane nominal molecular weight limit (NMWL) of 3 kDa. The centrifuged bacterial pellets are resuspended in initial volume of sterile water and are hereinafter referred to as heat-killed bacterial pellet solutions (HK-653-p, pellet).

To clarify the active ingredient is lipoteichoic acid (LTA) or peptidoglycan (PGN) in heat-killed bacterial pellets, the cell wall components, LTA or PGN, of heat-killed pellets of Lactobacillus paracasei GMNL-653 are further purified for following tests.

The process for purification of LTA is shown as follows. Heat-killed bacterial pellets are re-dissolved in 0.05 M sodium acetate (pH=4.0) and lyzed using an ultrasonic processor (model: W-220F). After centrifugation, the centrifuged bacterial pellets are re-dissolved in a mixed solution with chloroform, methanol and water (chloroform:methanol: water=1:1:0.9) for collecting the aqueous phase containing LTA. After residual methanol is evaporated, LTA is injected onto a column of Octyl-Sepharose CL-4B resins (2.5><20 cm; Sigma), previously equilibrated with a buffer containing 15% n-propyl alcohol and 0.05M sodium acetate (pH=4.7). LTA is eluted from the column with an increasing propyl alcohol gradient. The extracted LTA is subjected to a Q-Sepharose ion exchange column and refined by gradually increasing the concentration of NaCl, followed by the vacuum evaporation finally.

The process for purification of PGN is shown as follows. Heat-killed bacterial pellets are rinsed three times with phosphate buffered saline (PBS). Crude extracts are prepared from 50 g of lactobacillus (wet weight) lyzed by ultrasonication. By centrifugation of crude extracts (8,000 rpm; 30 minutes; 4° C.), pellets are collected, re-dissolved in distilled water (DW) and centrifuged again. The above step is repeated three times for collecting precipitates; the precipitates react with 100 mg/ml RNase as well as 50 mg/ml DNase for 18 hours, afterwards 200 mg/ml trypsin is added for another 18-hour co-reaction at 37° C.; the precipitates are centrifuged (8,000 rpm; 30 minutes; 4° C.) and freeze-dried. For preparation of PGN, 1 g of cell wall reacts with 5% trichloroacetic acid (TCA) for 18 hours at 22° C. and then is centrifuged (8,000 rpm; 30 minutes) for extractions of precipitates which are rinsed three times with water and acetone respectively, and then dried to get peptidoglycan powders.

[Clonogenic Assay]

Human nasopharyngeal carcinoma cells, NPC-BM1, and nasopharyngeal carcinoma cells, NPC-076, are planted in 12-well plates (100 cells/well), each of which is added by Lactobacillus paracasei GMNL-653 and heat-killed whole-bacterial liquids of Lactobacillus paracasei under the number of BCRC 910520, respectively. After 8-day cultivation, nasopharyngeal carcinoma cells are added by 2% formaldehyde for cell fixing, stained with 0.5% crystal violet and photographed for estimation of an area covered by nasopharyngeal carcinoma cells through ImageJ. With double-distilled water treating nasopharyngeal carcinoma cells as the control group, the ratio of an area covered by nasopharyngeal carcinoma cells treated with Lactobacillus paracasei GMNL-653 or heat-killed whole-bacterial liquids of Lactobacillus paracasei under the number of BCRC 910520 is calculated for analyzing the effect of Lactobacillus paracasei GMNL-653 or heat-killed whole-bacterial liquids of Lactobacillus paracasei under the number of BCRC 910520 on proliferations of nasopharyngeal carcinoma cells.

[Methylthiazol Tetrazolium Assay (MTT Assay)]

Human nasopharyngeal carcinoma cells, NPC-BM1, or nasopharyngeal carcinoma cells, NPC-076, are planted in 96-well plates (1000 cells/well) in which the following substances with distinct concentrations are added, respectively: heat-killed whole-bacterial liquids of Lactobacillus paracasei GMNL-653(“HK-653”); heat-killed bacterial pellets (“HK-653-p”, pellet) or separated heat-killed microbial supernatants (“HK-653-s”, sup); two portions of heat-killed microbial supernatants containing molecules with the molecular weight less than 3 kDa (“Sup<3K”) or with the molecular weight greater than 3 kDa (“Sup>3K”) separated by a centrifugal filter with 3 kDa cut-off; purified cell wall components of heat-killed bacterial pellets, LTA or PGN. With nasopharyngeal carcinoma cells cultivated for 48 or 72 hours, methylthiazol tetrazolium (MTT; 3-(4,5-dimethylthiazol-2-yl)-2,5-diphenyltetrazolium bromide; Sigma-Aldrich) is added and let incubate for 2 hours, afterwards the optical density (OD) at 570 nm is measured for evaluating cell survival and analyzing the effect of different portions of heat-killed whole-bacterial liquids of Lactobacillus paracasei GMNL-653 (that is, “HK-653”, “HK-653-p”, “HK-653-s”, “Sup<3K”, “Sup>3K”, “LTA” or “PGN”) on proliferations of nasopharyngeal carcinoma cells.

[Flow Cytometry for the Cell Cycle Assay]

Human nasopharyngeal carcinoma cells, NPC-BM1 and nasopharyngeal carcinoma cells, NPC-076, are planted into 3.5-cm culture dishes (1×10⁵ cells/dish) in which heat-killed whole-bacterial liquids (1×10⁹ cells/ml) of Lactobacillus paracasei GMNL-653 and an equal volume of double-distilled water (“control group”) are added, respectively. After 72 hours, nasopharyngeal carcinoma cells are detached from a 3.5-cm culture dish using trypsin/EDTA solutions and are collected by centrifugation (700 g; 6 minutes). After supernatants are removed, nasopharyngeal carcinoma cells are collected, fixed with 70% EtOH/PBS, and stored in a refrigerator at 4° C. overnight (>16 hours). Fixed nasopharyngeal carcinoma cells were centrifuged (700 g; 6 minutes) and supernatants are removed on the next day. Then, carcinoma cells are resuspended in 0.5 ml of PBS and stained with 300 μl of PI/RNase staining buffers (BD Pharmingen™; BD Biosciences) in the dark for a 30-minute incubation at room temperature. The stained cells are analyzed using the flow cytometer (BD FACSCalibur™; BD Biosciences) which detects fluorescence signals of propidium iodide (PI), and flow cytometry data are analyzed using FlowJo.

[Western Blotting]

Nasopharyngeal carcinoma cells, NPC-BM1, and nasopharyngeal carcinoma cells, NPC-076, are collected and added by NETN lysis buffers (100 mM NaCl, 20 mM Tris-Cl (pH=8.0), 0.5 mM EDTA and 0.5% (v/v) Nonidet P-40 (NP-40)) to lyse cells and extract proteins. The protein concentration is measured by the BCA (bicinchoninic acid) protein assay. 30 lug of proteins from each specimen is subjected to sodium dodecyl sulfate polyacrylamide gel electrophoresis (SDS-PAGE) and blotted on a PVDF film, followed by 1.5-hour blocking with 5% skim milk and incubation with specific primary antibodies at 4° C. overnight. On the next day, the PVDF films are incubated with horseradish peroxidase-conjugated secondary antibodies for 1 hour at room temperature. After rinsed properly, the PVDF films are added by chemiluminescent substrates and then chemiluminescent signals are detected and imaged by a Chemiluminescence imaging systems, followed by quantitative analysis of band intensities. The information for primary antibodies used in Western blotting is shown in Table 1.

TABLE 1

| Name of primary | | |
|---|---|---|
| antibody | Catalog number | Molecular weight |
| p-Rb (ser807/811) | 9308-Rabbit | 110 kDa, 1:1000 |
| CDK1 | GTX108120-Mouse | 34 kDa, 1:1000 |
| CDK2 | SC-6248-Mouse | 34 kDa, 1:1000 |
| CDK4 | SC-23896-Mouse | 34 kDa, 1:1000 |
| CDK6 | SC-7961-Mouse | 34 kDa, 1:1000 |
| Cyclin A2 | GTX103042-Rabbit | 50 kDa, 1:1000 |
| Cyclin B1 | GTX100911-Rabbit | 45 kDa, 1:1000 |
| Cyclin D1 | IR111-294-Rabbit | 34 kDa, 1:1000 |
| β-actin | A5441-Mouse | 42 kDa, 1:1000 |
| ATM | ab78-Mouse | 350 kDa, 1:1000 |
| p-ATM (ser1981) | ab36810-Mouse | 350 kDa, 1:1000 |
| mTOR | 66888-1-Ig-Mouse | 289 kDa, 1:1000 |
| p-mTOR (ser2448) | SC-293132-Mouse | 220 kDa, 1:1000 |
| AKT | GTX121937-Rabbit | 57 kDa, 1:1000 |
| p-AKT (ser473) | SC-135651-Rabbit | 56 kDa, 1:1000 |
| γ-H2AX (ser139) | GTX127340-Rabbit | 17 kDa, 1:1000 |
| γ-H2AX | GTX127340-Rabbit | 15 kDa, 1:1000 |
| Caspase-1 | GTX111630-Rabbit | 45 kDa, 1:1000 |
| GSDMD | ab155233-Rabbit | 53 kDa, 1:1000 |
| GAPDH | GTX100118-Rabbit | 35.8 kDa, 1:1000 |
| PTCH1 | 8358T kit #2468-Rabbit | 240 kDa, 1:1000 |
| PTCH2 | 8358T kit #2470-Rabbit | 130 kDa, 1:1000 |
| SUFU | 8358T kit #2520-Rabbit | 54 kDa, 1:1000 |
| GLI1 | 8358T kit #3538-Rabbit | 160 kDa, 1:1000 |
| Shh | 8358T kit #2207-Rabbit | 19 kDa, 1:1000 |

[Measurement of Mitochondrial Membrane Potential]

Human nasopharyngeal carcinoma cells, NPC-BM1, and nasopharyngeal carcinoma cells, NPC-076, each of which are treated with heat-killed whole-bacterial liquids (1×10⁹ cells/ml) of Lactobacillus paracasei GMNL-653 or Cisplatin (5 μM; “positive control”), cultivated for 48 hours and stained by JC-1 dyes (Cat. No. 551302; BD Pharmingen™) Then, the change in the mitochondrial membrane potential is measured by flow cytometry for analyzing the effect of heat-killed whole-bacterial liquids of Lactobacillus paracasei GMNL-653 on the mitochondrial membrane potential.

[Quantification of Reactive Oxygen Species (ROS)]

Nasopharyngeal carcinoma cells, NPC-BM1, and nasopharyngeal carcinoma cells, NPC-076, are added by heat-killed whole-bacterial liquids (“HK-653”) of Lactobacillus paracasei GMNL-653 and cultivated in 6-cm culture dishes for 24 hours, respectively. The nasopharyngeal carcinoma cells detached by trypsin are collected and then dispensed to 5 ml centrifuge tubes (1.5×10⁵ cells/ml/tube) for centrifugation (500 G; 5 minutes), followed by staining with 10 μM DCFDA dyes (Cat. No. 601520; Cayman Chemical) for 30-minute at 37° C. Then, heat-killed whole-bacterial liquids (1×10⁹ cells/ml) of Lactobacillus paracasei GMNL-653 or H₂O₂ (350 μM; “positive control”) are added for 30-minute incubation at 37° C. followed by centrifugation (400 G; 5 minutes). With nasopharyngeal carcinoma cells resuspended in 300 μl of PBS, single-cell fluorescence signals are detected by flow cytometry (BD FACSCalibur™; BD Biosciences), and fluorescent signals of cells are quantified by FlowJo for evaluating whether heat-killed whole-bacterial liquids of Lactobacillus paracasei GMNL-653 induce ROS production of nasopharyngeal carcinoma cells.

[Detection of Activity of Nasopharyngeal Carcinoma Stein Cells]

Human nasopharyngeal carcinoma cells, NPC-BM1, and nasopharyngeal carcinoma cells, NPC-076, are cultivated in ultra-low attachment culture dishes (Cat. No. 657185; Greiner Bio-One GmbH, Austria) and maintained in serum-free DMEM/F12 media with additional supplements such as 20 ng/ml epidermal growth factor (EGF; Cat. No. AF-100-15; PeproTech, Inc.), 20 ng/ml basic fibroblast growth factor (bFGF; Cat. No. 100-18B; PeproTech, Inc.), 0.5×B27 supplement (Cat. No. 17504044; Gibco™, Thermo Fisher Scientific), 1 μM hydrocortisone (Cat. No. H0888; Sigma-Aldrich), 5 μg/ml insulin (Cat. No. 13536; Sigma-Aldrich), 4 μg/ml heparin (Cat. No. H3149; Sigma-Aldrich) for cultivation of tumor spheroids and analysis of stein cell activities in nasopharyngeal carcinoma cells, NPC-BM1, and nasopharyngeal carcinoma cells, NPC-076. Then, heat-killed whole-bacterial liquids (1×10⁸ or 1×10⁹ cells/ml) of Lactobacillus paracasei GMNL-653 are added into culture dishes for 10-12 day cultivation, and afterwards the number of tumor spheroids formed from nasopharyngeal carcinoma cells, NPC-BM1, or nasopharyngeal carcinoma cells, NPC-076, are determined and photographed with an inverted microscope for analyzing the effect of heat-killed whole-bacterial liquids of Lactobacillus paracasei GMNL-653 on self-renewal activity of nasopharyngeal carcinoma cells.

[Statistic Analysis]

The data are presented by “Mean±SD”. The significant differences between two groups and between multiple groups are analyzed with the Student's t-test and the one-way analysis of variance (one-way ANOVA), respectively. For statistic results, p<0.05 denotes a meaningful difference.

### Embodiment 1

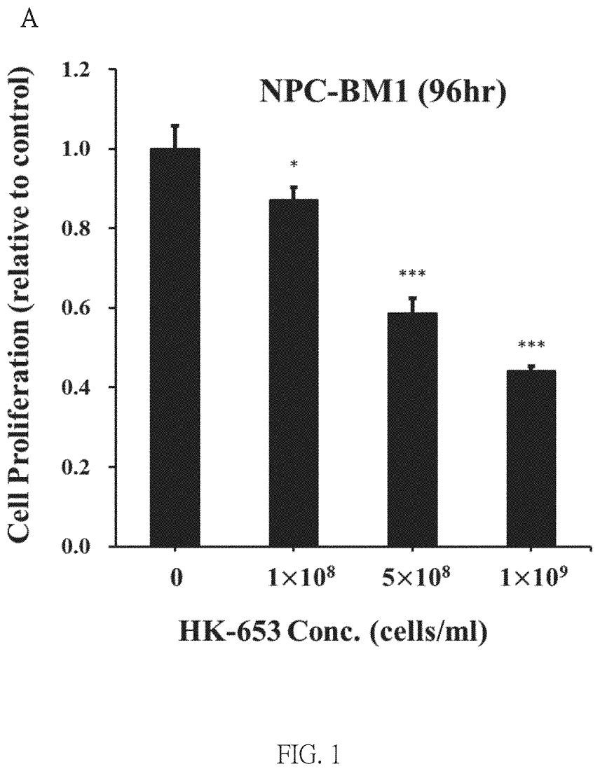

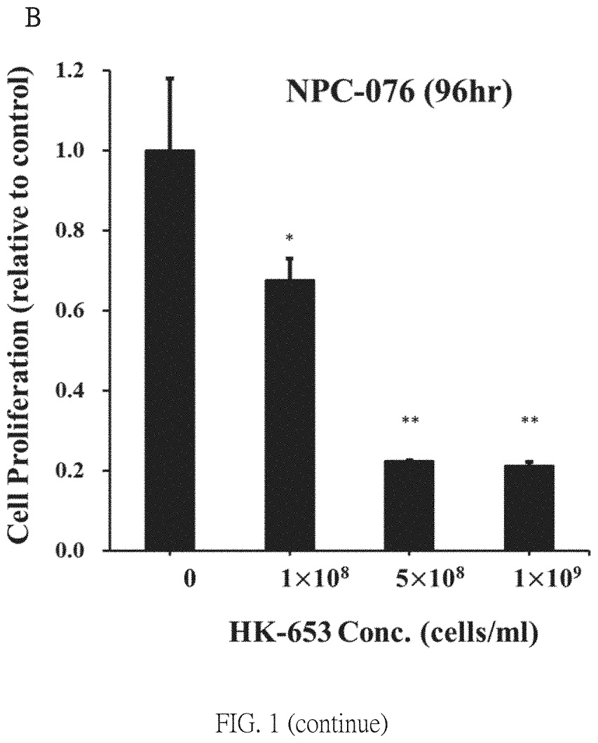

Referring to FIG. 1, which illustrates the effect of heat-killed whole-bacterial liquids (HK-653) of Lactobacillus paracasei GMNL-653 on proliferations of nasopharyngeal carcinoma cells according to the Methylthiazol tetrazolium assay (MTT assay). Nasopharyngeal carcinoma cells, NPC-BM1, (A of FIG. 1) and nasopharyngeal carcinoma cells, NPC-076, (B of FIG. 1) are treated with heat-killed whole-bacterial liquids (1×10⁸, 5×10⁸ and 1×10⁹ cells/ml; diluted in sterile double-distilled water) of Lactobacillus paracasei GMNL-653, respectively. After 96 hours, nasopharyngeal carcinoma cells are added by MTT and let incubate for two hours, and afterwards the optical density (OD) at 570 nm is measured for evaluating cell survivals. In statistic analysis, nasopharyngeal carcinoma cells treated with heat-killed whole-bacterial liquids at zero concentration are the control groups, and the relative proliferation ratio for each treatment is calculated relative to the optical density of the control group (“*”: p<0.05; “**”: p<0.01; “***”: p<0.001). As shown in test results, proliferations of nasopharyngeal carcinoma cells are inhibited with an increasing concentration of heat-killed whole-bacterial liquids of Lactobacillus paracasei GMNL-653.

### Embodiment 2

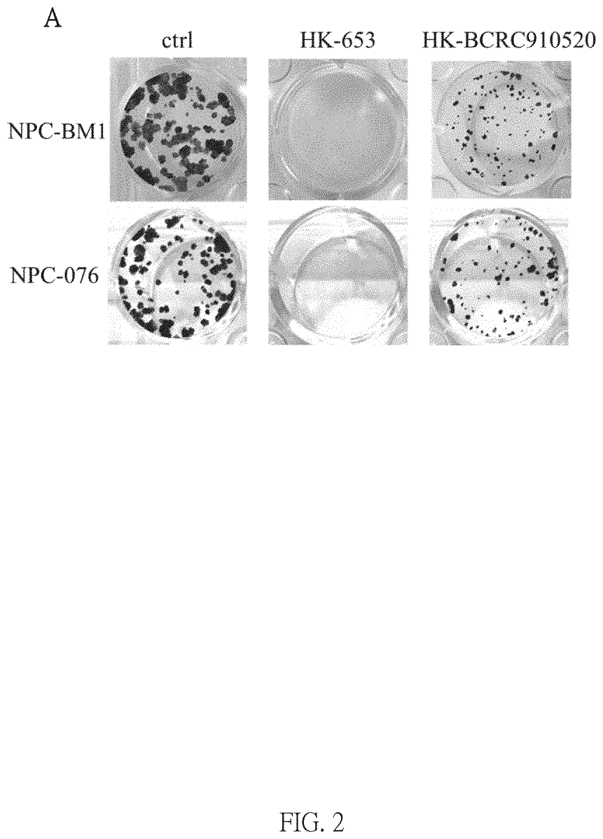

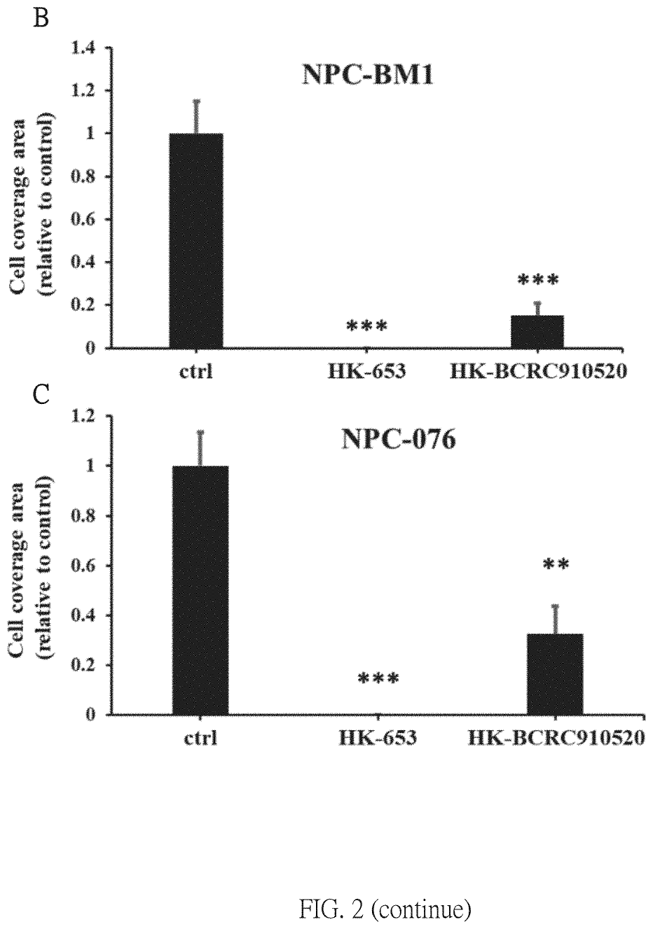

Referring to FIG. 2, which illustrates the effect of heat-killed whole-bacterial liquids (HK-653) of Lactobacillus paracasei GMNL-653 on proliferations of nasopharyngeal carcinoma cells according to the clonogenic assay. A of FIG. 2 illustrates appearances of nasopharyngeal carcinoma cells cultivated in culture dishes; B of FIG. 2 and C of FIG. 2 illustrate quantitative results of nasopharyngeal carcinoma cells, NPC-BM1, and nasopharyngeal carcinoma cells, NPC-076, respectively. In the clonogenic assay, a small quantity of nasopharyngeal carcinoma cells are planted in a 12-well plate (100 cells/well), treated with heat-killed whole-bacterial liquids of Lactobacillus paracasei, and then colony formation are monitored after eight day cultivation. As shown in B and C of FIG. 2, “ctrl”, “HK-653” and “HK-BCRC 910520” mean “control group” based on double-distilled water (100 μl/well) for treatment, heat-killed whole-bacterial liquids of Lactobacillus paracasei GMNL-653 (concentration: 1×10⁹ cells/ml) and heat-killed whole-bacterial liquids of Lactobacillus paracasei under the number of BCRC 910520 (concentration: 1×10⁹ cells/ml), respectively. In statistic analysis, the status for proliferations of nasopharyngeal carcinoma cells is expressed as the cell coverage area ratio calculated relative to those of the control group (“**”: p<0.01; “***”: p<0.001). As shown in test results, long-term proliferations of nasopharyngeal carcinoma cells are obviously inhibited by Lactobacillus paracasei GMNL-653 which is significantly better than Lactobacillus paracasei under the number of BCRC 910520.

### Embodiment 3

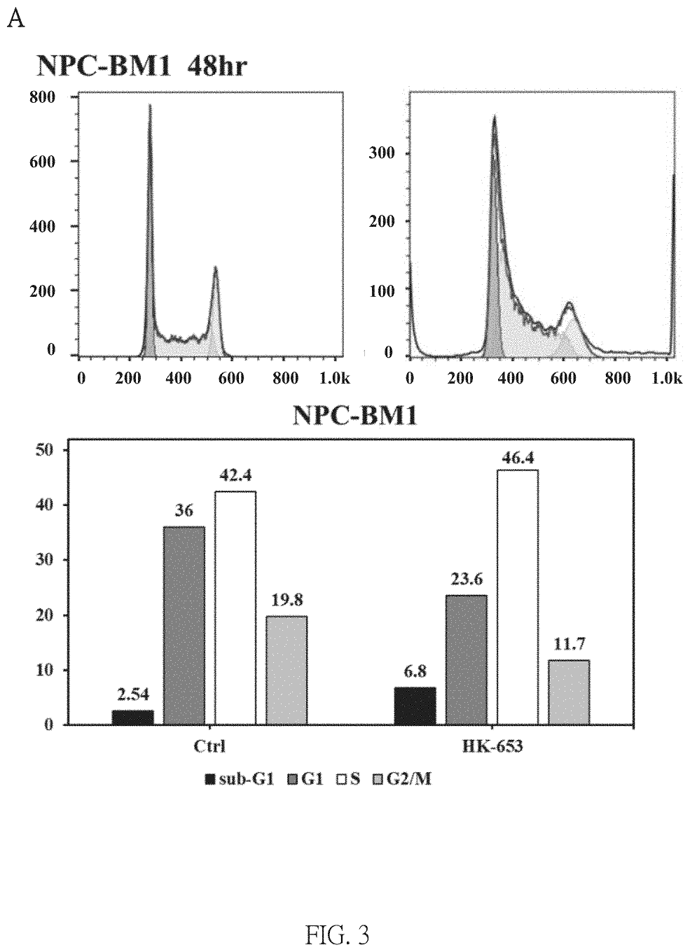

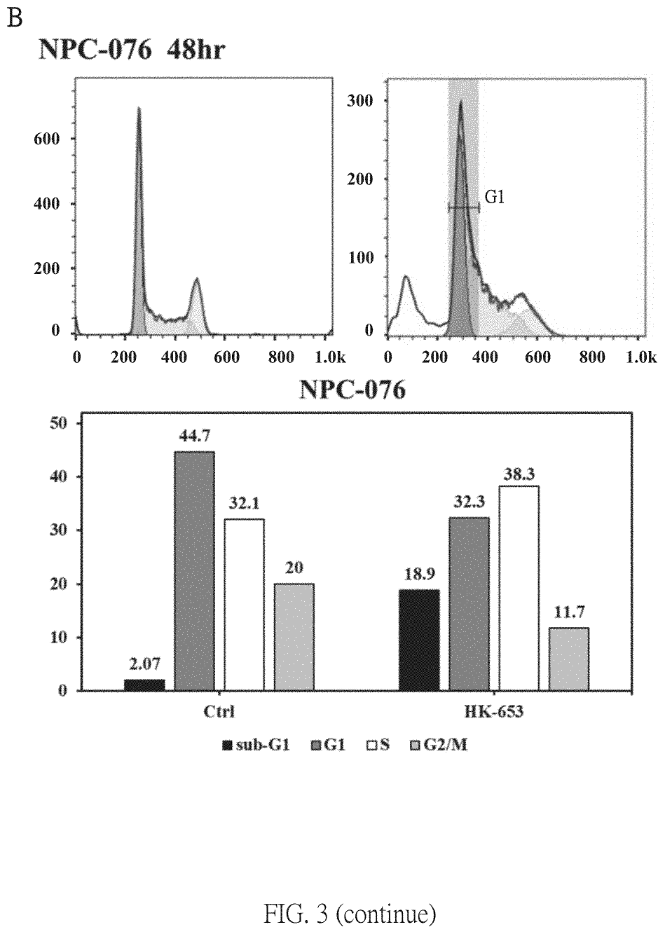

In Embodiment 3, a hypothesis that proliferations of nasopharyngeal carcinoma cells inhibited are attributed to cell deaths or disturbed cell cycle progression induced by Lactobacillus paracasei GMNL-653 is explored. For that matter, the propidium iodide (PI) staining is used in analyzing the effect of Lactobacillus paracasei GMNL-653 on the cell cycle of nasopharyngeal carcinoma cells. Referring to FIG. 3, which illustrates the effect of heat-killed whole-bacterial liquids (HK-653) of Lactobacillus paracasei GMNL-653 on the cell cycle of nasopharyngeal carcinoma cells. Nasopharyngeal carcinoma cells, NPC-BM1, (A of FIG. 3) and nasopharyngeal carcinoma cells, NPC-076, (B of FIG. 3) are treated with heat-killed whole-bacterial liquids (HK-653) of Lactobacillus paracasei GMNL-653 for 48 hours, then stained with PI dyes and subjected to flow cytometry for fluorescence signal detection, followed by data analysis with FlowJo. It can be seen from test results that the percentage of nasopharyngeal carcinoma cells, NPC-BM1, treated with Lactobacillus paracasei GMNL-653 for 48 hours decreases from 36% to 23.6% in the G1 phase and from 19.8% to 11.7% in the G2/M phase, increases from 2.54% to 6.8% in the sub-G1 phase and stagnates in the S phase (from 42.4% to 46.4%). Similarly, the percentage of nasopharyngeal carcinoma cells, NPC-076, treated with Lactobacillus paracasei GMNL-653 for 48 hours increases from 2.07% to 18.9% in the sub-G1 phase significantly and from 32.1% to 38.3% in the S phase slightly.

### Embodiment 4

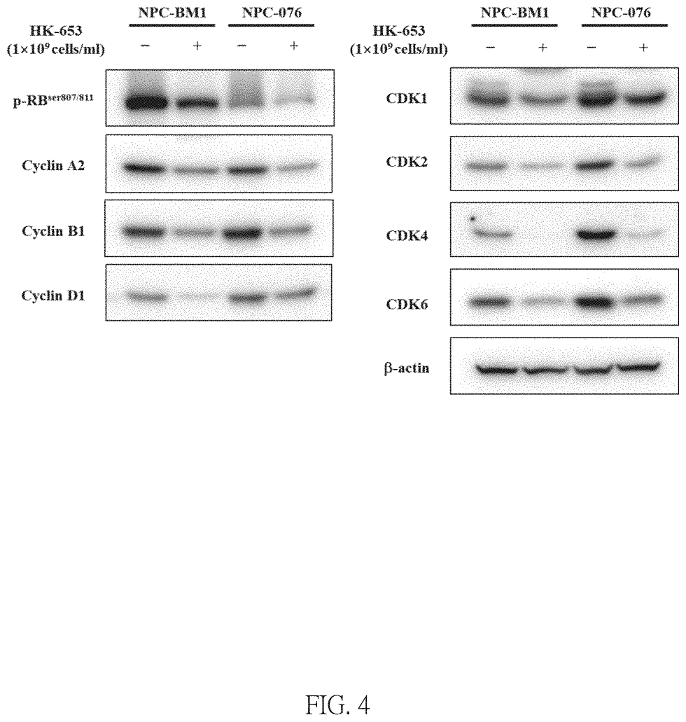

Referring to FIG. 4, which illustrates the effect of heat-killed whole-bacterial liquids (HK-653) of Lactobacillus paracasei GMNL-653 on expressions of proteins related to a cell cycle of nasopharyngeal carcinoma cells. Nasopharyngeal carcinoma cells are treated with heat-killed whole-bacterial liquids (1×10⁹ cells/ml) of Lactobacillus paracasei GMNL-653 for 48 hours, then total cellular proteins are collected and protein expressions are analyzed by Western blotting in which β-actin is used as a loading control to normalize the levels of protein. As shown in test results, expressions of proteins related to the cell cycle including p-RBser^{807/811}, Cyclin A2, Cyclin B1, Cyclin D1, CDK1, CDK2, CDK4 and CDK6, are inhibited in nasopharyngeal carcinoma cells treated with heat-killed whole-bacterial liquids of Lactobacillus paracasei GMNL-653, that is, proliferations of nasopharyngeal carcinoma cells slow down due to a change in the cell cycle distribution of nasopharyngeal carcinoma cells induced by Lactobacillus paracasei GMNL-653.

In summary, Lactobacillus paracasei GMNL-653 inhibits proliferations of nasopharyngeal carcinoma cells or reduces a probability of developing nasopharyngeal carcinoma by inducing the cell cycle arrest of nasopharyngeal carcinoma cells.

### Embodiment 5

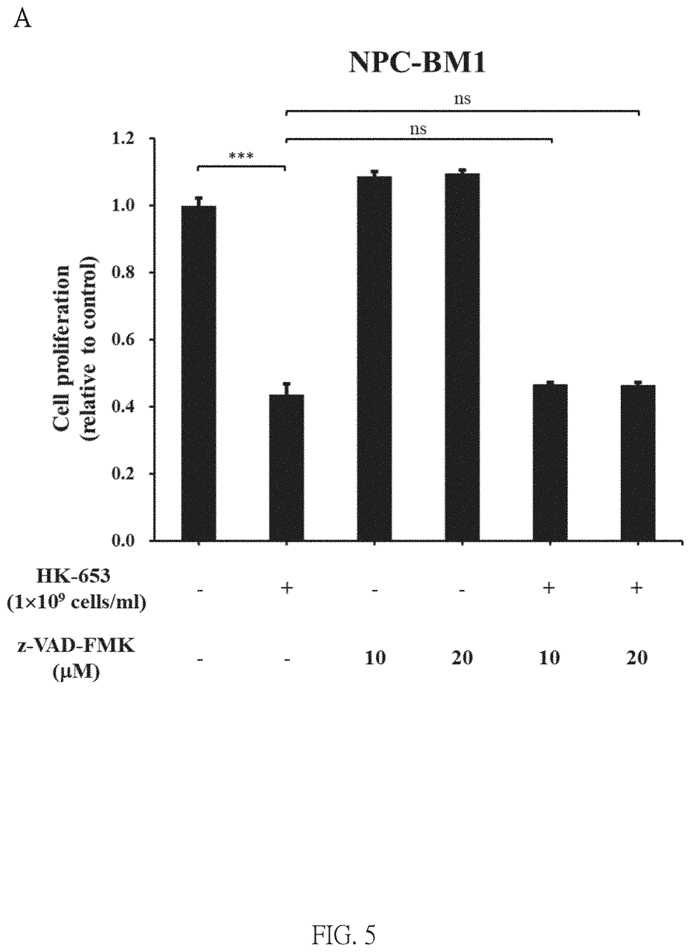

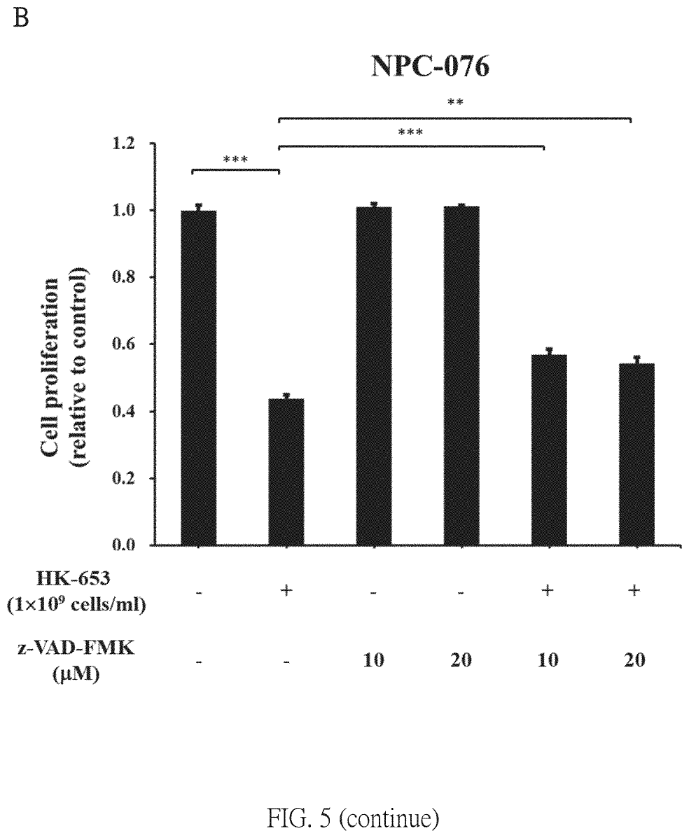

In the present disclosure, the fact that the percentage of nasopharyngeal carcinoma cells increases in the sub-G1 phase has been disclosed in the previous embodiment. In Embodiment 5, the topic of evaluating apoptosis as a probable mechanism for heat-killed whole-bacterial liquids of Lactobacillus paracasei GMNL-653 inhibiting proliferations of nasopharyngeal carcinoma cells is explored. Two nasopharyngeal carcinoma cell lines which are treated with heat-killed whole-bacterial liquids (1×10⁹ cells/ml) of Lactobacillus paracasei GMNL-653 as well as a broad-spectrum caspase inhibitor of apoptosis, z-VAD-FMK (10 μm; 20 μm), cultivated for 72 hours, and then proliferations of carcinoma cells are analyzed with the MTT assay. Referring to FIG. 5, which illustrates the effect of heat-killed whole-bacterial liquids (HK-653) of Lactobacillus paracasei GMNL-653 on apoptosis of nasopharyngeal carcinoma cells, NPC-BM1, (A of FIGS. 5) and nasopharyngeal carcinoma cells, NPC-076, (B of FIG. 5). The status for proliferations of nasopharyngeal carcinoma cells is expressed as the relative proliferation ratio for each treatment calculated relative to those of the carcinoma cells treated with neither heat-killed whole-bacterial liquids nor z-VAD-FMK (“control group”) (“**”: p<0.01; “***”: p<0.001; “ns”: no significant difference). As shown in test results, proliferations of two nasopharyngeal carcinoma cell lines are significantly inhibited by heat-killed whole-bacterial fluids of Lactobacillus paracasei GMNL-653 even in the presence of apoptosis inhibitor, z-VAD-FMK, that is, proliferations of nasopharyngeal carcinoma cells inhibited by heat-killed whole-bacterial liquids of Lactobacillus paracasei GMNL-653 are not attributed to apoptosis.

In summary, dissimilar to the traditional mechanism based on probiotics to inhibit proliferations of carcinoma cells, Lactobacillus paracasei GMNL-653 reduces proliferations of nasopharyngeal carcinoma cells or a probability for development of nasopharyngeal carcinoma should not be attributed to apoptosis.

### Embodiment 6

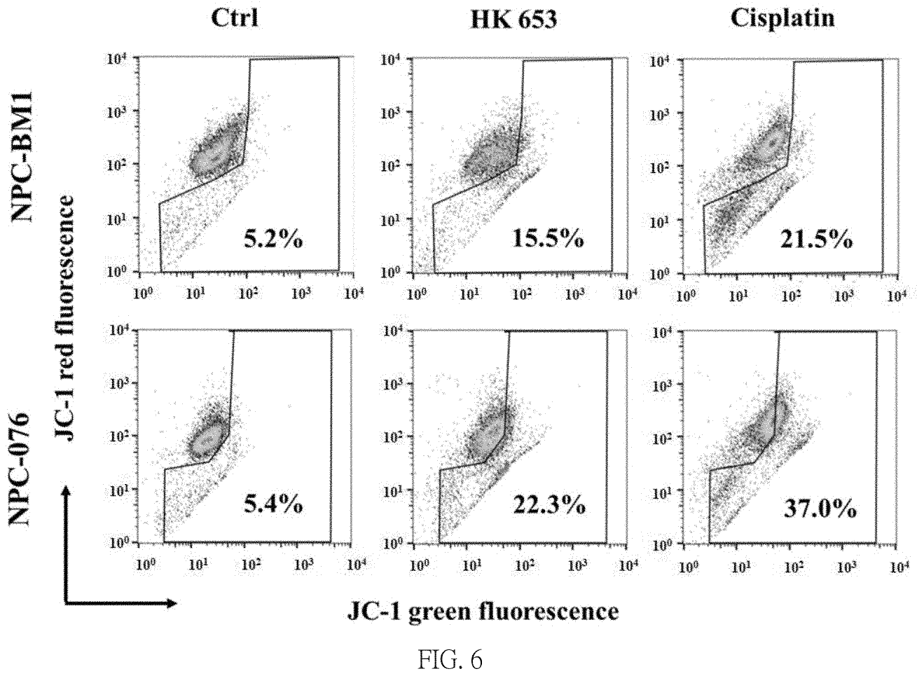

As shown in test results in FIG. 3, Lactobacillus paracasei GMNL-653 increases the propotion of nasopharyngeal carcinoma cells in the sub-G1 phase of cell cycle that are probably caused by a decrease in mitochondrial membrane potential, leading to cell death of carcinoma cells. With Cisplatin (5 μM) applied on nasopharyngeal carcinoma cells (as the “positive control group”) in Embodiment 6, carcinoma cells are stained with JC-1 dyes to recognize the status of a decrease in mitochondrial membrane potential. Referring to FIG. 6, which illustrates the effect of heat-killed whole-bacterial liquids (HK-653) of Lactobacillus paracasei GMNL-653 on a mitochondrial membrane potential in nasopharyngeal carcinoma cells. Nasopharyngeal carcinoma cells treated with heat-killed whole-bacterial liquids (HK-653) of Lactobacillus paracasei GMNL-653 for 48 hours are stained with JC-1 dyes and subjected to flow cytometry for red fluorescence signals (cells displaying a normal mitochondrial membrane potential) or green fluorescence signals (cells displaying a decreased mitochondrial membrane potential) detection, followed by data analysis with WinMDI. As shown in FIG. 6, “ctrl”, “HK-653” and “Cisplatin” mean “control group” based on double-distilled water for treatment, heat-killed whole-bacterial liquids (concentration: 1×10⁹ cells/ml) of Lactobacillus paracasei GMNL-653 and the “positive control group” (concentration of cisplatin: 5 μM), respectively. It can be seen from test results that a decrease in mitochondrial membrane potential in each of two nasopharyngeal carcinoma cell lines is definitely attributed to Lactobacillus paracasei GMNL-653.

### Embodiment 7

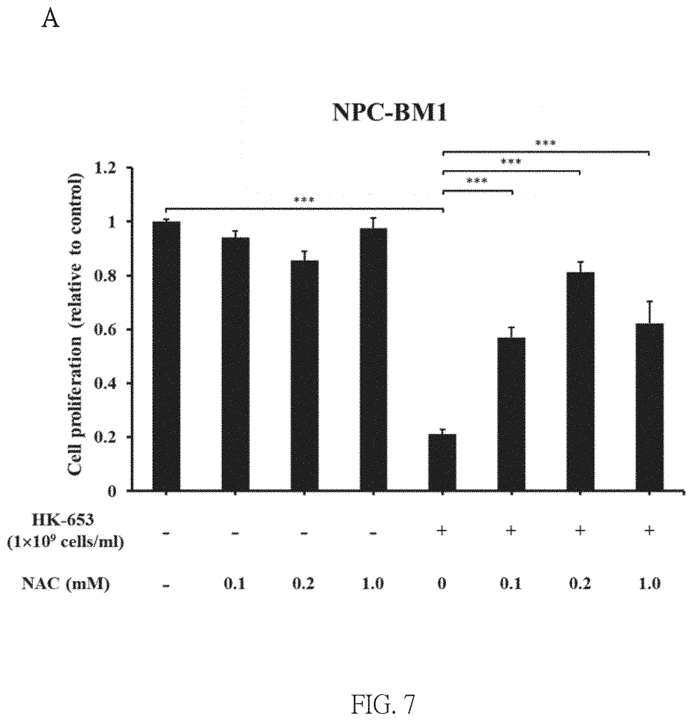

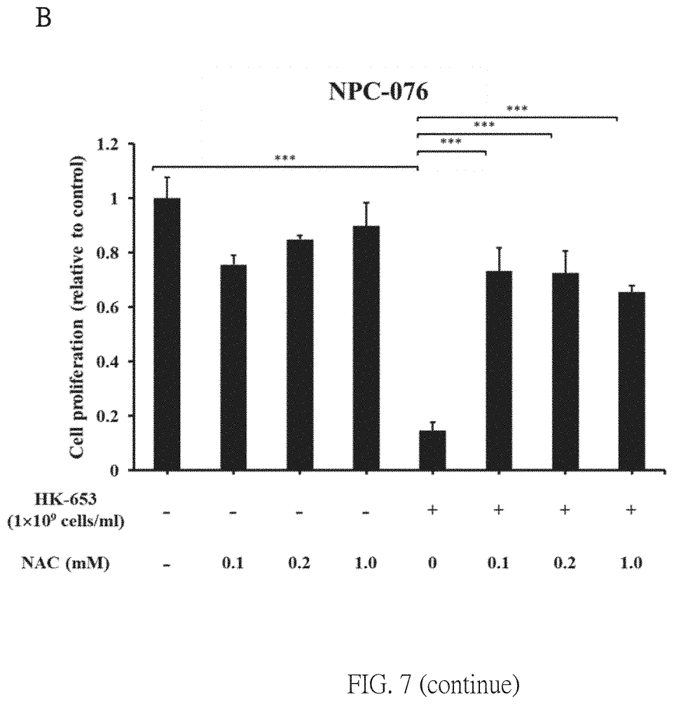

Reactive oxygen species (ROS) generated in cells may be one of the reasons why mitochondrial membrane potential is reduced, resulting in inhibited proliferations of cells. In Embodiment 7, N-acetyl-1-cysteine (NAC), a broad-spectrum ROS scavenger, is used in exploring the probable role of ROS for heat-killed whole-bacterial liquids of Lactobacillus paracasei GMNL-653 inhibiting proliferative activity of nasopharyngeal carcinoma cells. Referring to FIG. 7, which illustrates the effect of ROS inhibitors as well as heat-killed whole-bacterial liquids (HK-653) of Lactobacillus paracasei GMNL-653 on proliferations of nasopharyngeal carcinoma cells. Proliferations of nasopharyngeal carcinoma cells, NPC-BM1, (A of FIG. 7) and nasopharyngeal carcinoma cells, NPC-076, (B of FIG. 7), both of which are treated with HK-653 (1×10⁹ cells/ml) and/or NAC (0.1 mM; 0.2 mM; 1.0 mM) for 72 hours, and afterwards are evaluated with the MTT assay. The status for proliferations of nasopharyngeal carcinoma cells is expressed as the ratio for each treatment calculated relative to those of the carcinoma cells treated with neither NAC nor heat-killed whole-bacterial liquids (“control group”) (“***”: p<0.001). After two nasopharyngeal carcinoma cell lines are treated with NAC as well as heat-killed whole-bacterial liquids of Lactobacillus paracasei GMNL-653, heat-killed whole-bacterial liquids of Lactobacillus paracasei GMNL-653 fall short of the efficiency to inhibit proliferations of nasopharyngeal carcinoma cells significantly. Accordingly, inhibition of nasopharyngeal carcinoma cell proliferation is attributed to the accumulation of ROS in nasopharyngeal carcinoma cells treated with heat-killed whole-bacterial liquids of Lactobacillus paracasei GMNL-653.

### Embodiment 8

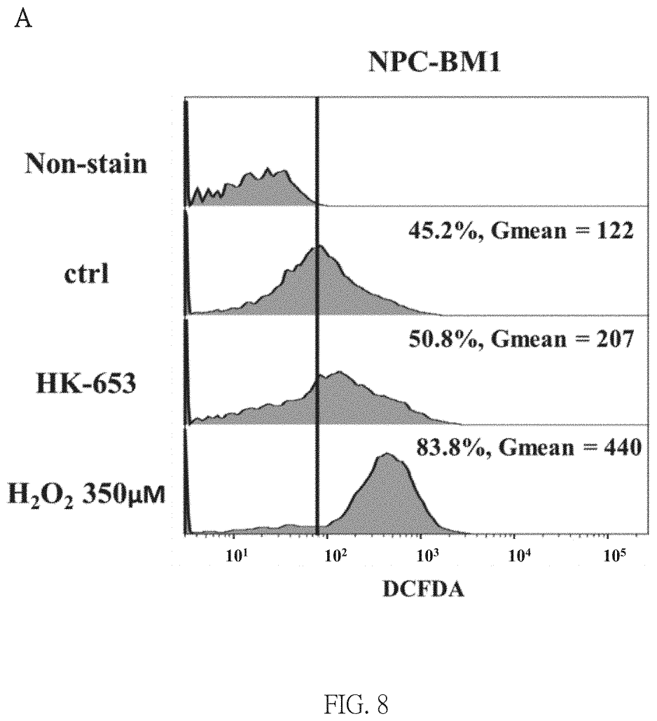

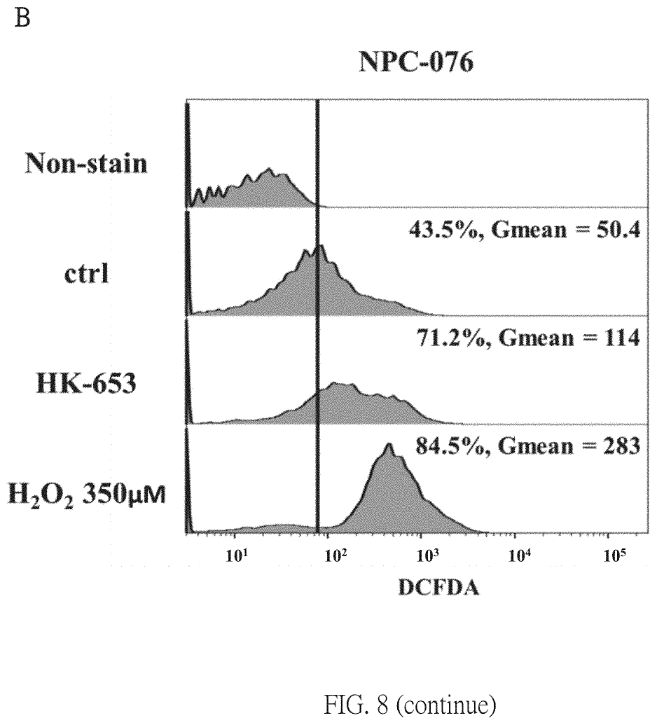

In Embodiment 8, the level of ROS is measured with DCFDA dyes through which the fact that ROS is accumulated in two nasopharyngeal carcinoma cell lines treated with heat-killed whole-bacterial liquids of Lactobacillus paracasei GMNL-653 is verified. Referring to FIG. 8, which illustrates the effect of heat-killed whole-bacterial liquids (HK-653) of Lactobacillus paracasei GMNL-653 on accumulation of ROS in nasopharyngeal carcinoma cells. Nasopharyngeal carcinoma cells, NPC-BM1, (A of FIG. 8) and nasopharyngeal carcinoma cells, NPC-076, (B of FIG. 8), both of which are marked by DCFDA and treated with some additives at 37° C. for 30 minutes, are measured with a flow cytometer for detection of fluorescence signals and quantitative data analysis with FlowJo. In FIG. 8, “Non-stain”, “ctrl”, “HK-653” and “H₂O₂ 350 μM” mean nasopharyngeal carcinoma cells not marked by DCFDA (that is, the fluorescent background value of cells), nasopharyngeal carcinoma cells marked by DCFDA but untreated, heat-killed whole-bacterial liquids (1×10⁹ cells/ml) of Lactobacillus paracasei GMNL-653 treated, and H₂O₂ (concentration: 350 μM) treated, respectively; “GMean” is the geometric mean. As shown in test results for the level of fluorescence analyzed by flow cytometry, ROS is significantly generated in nasopharyngeal carcinoma cells which have been treated with heat-killed whole-bacterial liquids of Lactobacillus paracasei GMNL-653.

### Embodiment 9

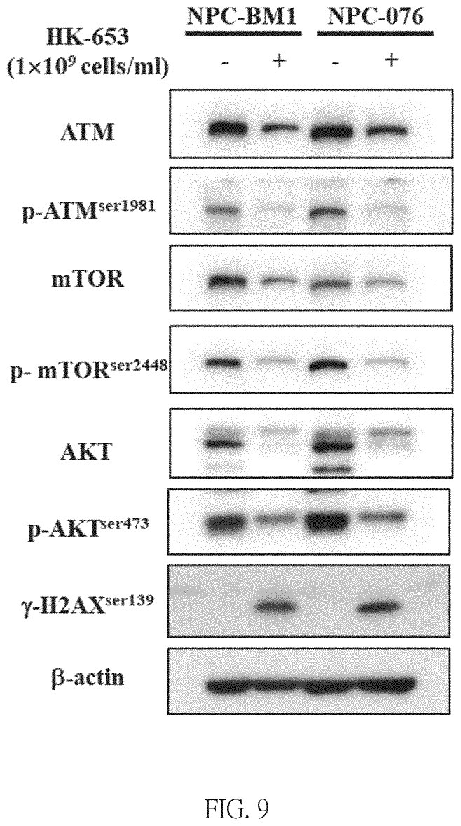

For nasopharyngeal carcinoma cells treated with heat-killed whole-bacterial liquids of Lactobacillus paracasei GMNL-653, the expressions of proteins for DNA damages induced by ROS are further analyzed in Embodiment 9. Referring to FIG. 9, which illustrates the effects of heat-killed whole-bacterial liquids (HK-653) of Lactobacillus paracasei GMNL-653 on proteins related to DNA damages and DNA repair pathways in nasopharyngeal carcinoma cells. After nasopharyngeal carcinoma cells are treated for 48 hours, the expressions of relative proteins are analyzed by Western blotting in which β-actin is used as a loading control to normalize the levels of protein. As shown in test results, the level of phosphorylated γH2AX^{ser139} which induces DNA damage pathways increases significantly after nasopharyngeal carcinoma cells are treated with heat-killed whole-bacterial liquids of Lactobacillus paracasei GMNL-653. However, the expressions of the DNA repair proteins ATM and p-ATM^{ser1981} decrease, demonstrating the activations of DNA damage pathways but inhibitions of DNA repair pathways in nasopharyngeal carcinoma cells. Moreover, activation of the Akt/mTOR pathway involved in cell proliferations is also inhibited.

### Embodiment 10

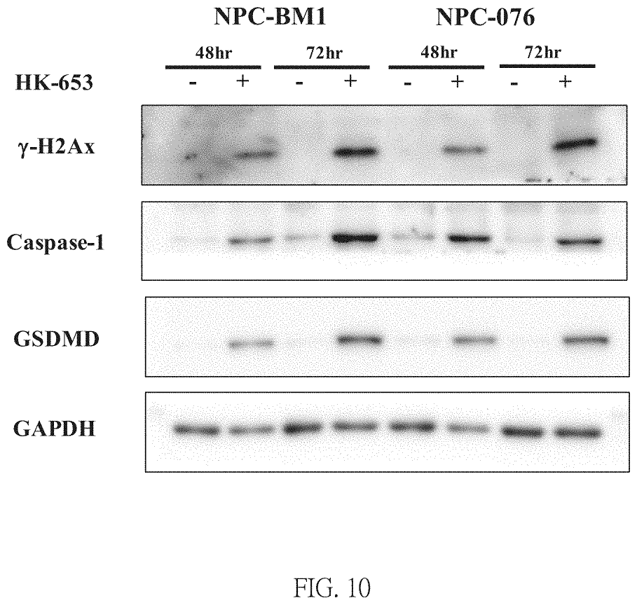

Pyroptosis which is induced by Caspase-1 after inflammasome activation is defined as a type of programmed cell death. Pyroptosis refers to a process in which the pores are formed on cell membranes first, cells gradually swell until the membrane rupture, the cellular contents are released into extracellular space and then trigger intense inflammatory responses. As the innate immune response of an organism, pyroptosis discovered in innate immune cells as well as in tumors has been considered as a promising strategy for cancer therapy. In addition to accumulation of ROS and DNA damages in cells mentioned previously, other features of pyroptosis are expressions of pyroptosis-related proteins such as Caspase-1 and gasdermin D (GSDMD) that will be analyzed with Western blotting. Referring to FIG. 10, which illustrates the effect of heat-killed whole-bacterial liquids (HK-653) of Lactobacillus paracasei GMNL-653 on pyroptosis-related proteins in nasopharyngeal carcinoma cells and β-actin is used as a loading control to normalize the levels of protein. As shown in test results, the pyroptosis pathway of nasopharyngeal carcinoma cells treated with heat-killed whole-bacterial liquids of Lactobacillus paracasei GMNL-653 is activated in virtue of significantly increasing expressions of γH2AX related to DNA damages, Caspase-1 with molecules critical to pyroptosis, and GSDMD, a pore-forming protein related to cell swelling and lysis.

In summary, pyroptosis of nasopharyngeal carcinoma cells activated by Lactobacillus paracasei GMNL-653 inhibits proliferations of nasopharyngeal carcinoma cells or the probability of nasopharyngeal carcinoma development because of some characteristics activating pyroptosis such as accumulation of ROS in cells, DNA damages, and Caspase-1 as well as GSDMD activation.

### Embodiment 11

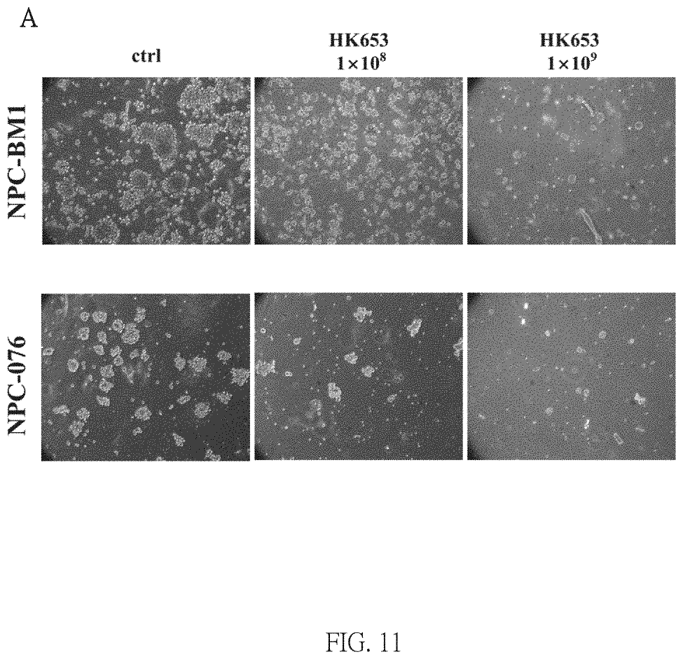

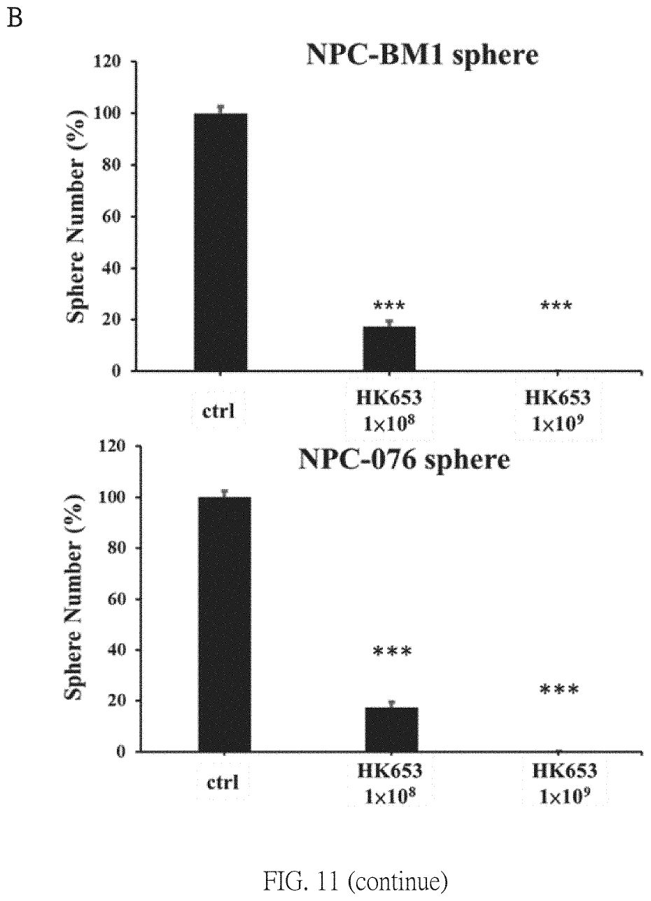

Cancer stein cells featuring high carcinogenicity, resistance to drugs and metastasis are considered as the subpopulation to be targeted within tumors and the activity of cancer stein cells is evaluated by tumor spheroid assay effectively. Referring to FIG. 11, which illustrates the effect of heat-killed whole-bacterial liquids (HK-653) of Lactobacillus paracasei GMNL-653 on activity of nasopharyngeal carcinoma stein cells; A of FIG. 11 illustrates appearances of tumor spheroids grown from nasopharyngeal carcinoma cells; B of FIG. 11 illustrates quantitative test results for tumor spheroids grown from nasopharyngeal carcinoma cells, NPC-BM1, and nasopharyngeal carcinoma cells, NPC-076. In Embodiment 11, the cancer stein cell activity of two nasopharyngeal carcinoma cell lines treated with heat-killed whole-bacterial liquids of Lactobacillus paracasei GMNL-653 is analyzed through cultivation of tumor spheroids. As shown in FIG. 11, “ctrl” and “HK-653” mean “control group” based on double-distilled water (20 μl/well) for treatment and heat-killed whole-bacterial liquids (concentration: 1×10⁸ cells/ml or 1×10⁹ cells/nil) of Lactobacillus paracasei GMNL-653, respectively. In statistic analysis, the status of cancer stein cell activity is expressed as the relative percentage of formed tumor spheroids in carcinoma cells treated with heat-killed whole-bacterial liquids calculated relative to those of control group (“***”: p<0.001); the magnification of 100× is designated to each photo. It can be seen from test results that the capability of developing tumor spheroids in nasopharyngeal carcinoma cells, NPC-BM1, or nasopharyngeal carcinoma cells, NPC-076, are inhibited by Lactobacillus paracasei GMNL-653 in a concentration-dependent manner, suggesting self-renewal ability of nasopharyngeal carcinoma stein cells are diminished by Lactobacillus paracasei GMNL-653.

### Embodiment 12

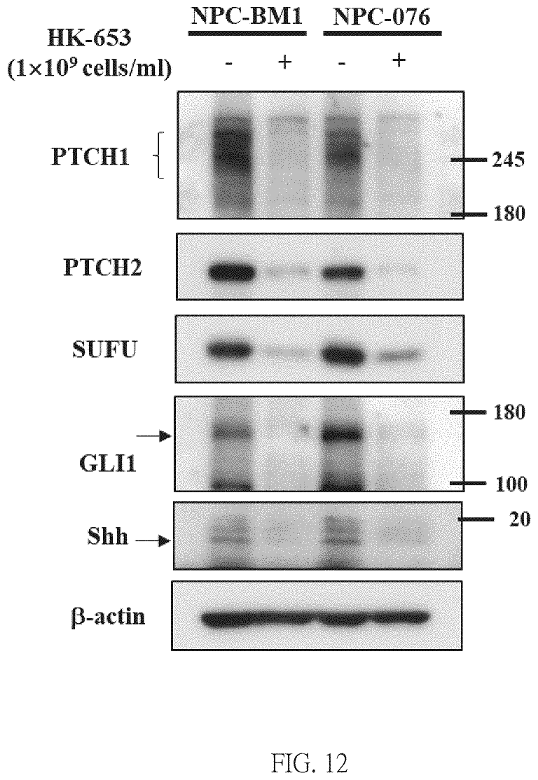

Self-renewals of cancer stein cells are probably correlated with activation of the Hedgehog pathway. With protein Sonic hedgehog (Shh) and its receptor Patched (Ptch1) binding each other, the message of activating protein Smo, enabling protein GLI to enter cell nuclei and activating expression of downstream genes is initiated. Tumor development and metastasis is probably attributed to the over-expression of Shh. As disclosed in previous literatures, the activity for self-renewals of nasopharyngeal carcinoma stein cells is weakened when the Shh signal is inhibited. In Embodiment 12, nasopharyngeal carcinoma cells, NPC-BM1, or nasopharyngeal carcinoma cells, NPC-076, are treated with heat-killed whole-bacterial liquids of Lactobacillus paracasei GMNL-653 and the expressions of proteins related to the SHH pathway, i.e., PTCH1, PTCH2, SUFU, GLI1 and Shh, are analyzed with Western blotting in which β-actin is used as a loading control to normalize the levels of protein. Referring to FIG. 12, which illustrates the effect of heat-killed whole-bacterial liquids (HK-653) of Lactobacillus paracasei GMNL-653 on proteins related to the SHH pathway (for self-renewals of nasopharyngeal carcinoma stein cells). The two nasopharyngeal carcinoma cell lines are treated with heat-killed whole-bacterial liquids for 48 hours, and the expressions of above proteins are analyzed with Western blotting. As shown in test results, the expressions of proteins related to the self-renewal pathway of nasopharyngeal carcinoma stein cells are inhibited. In other words, with the SHH pathway inhibited by heat-killed whole-bacterial liquids of Lactobacillus paracasei GMNL-653, the capability for self-renewals of nasopharyngeal carcinoma stein cells is diminished, resulting in a low probability of tumor development.

In summary, Lactobacillus paracasei GMNL-653 reduces proliferations of nasopharyngeal carcinoma cells or probable tumor development by inhibiting self-renewals of nasopharyngeal carcinoma stein cells.

### Embodiment 13

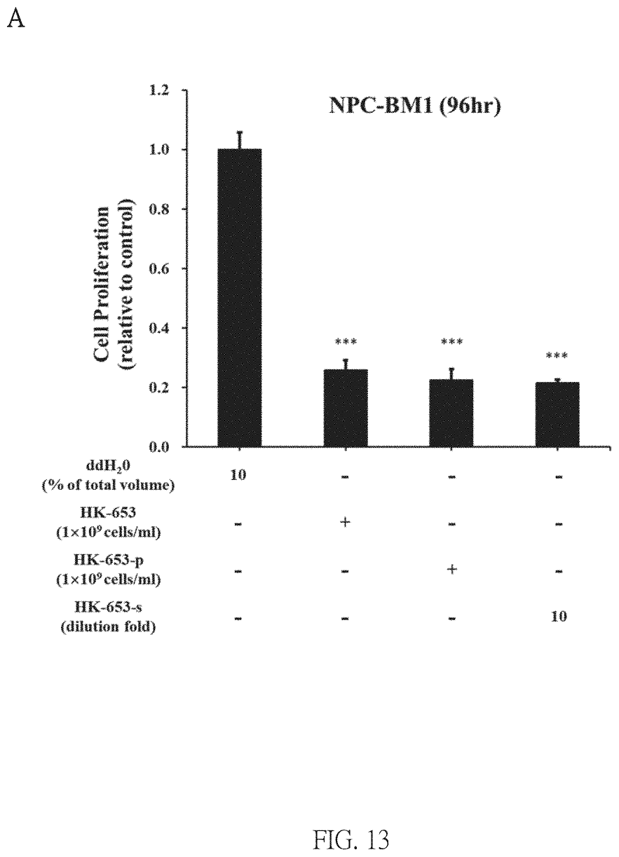

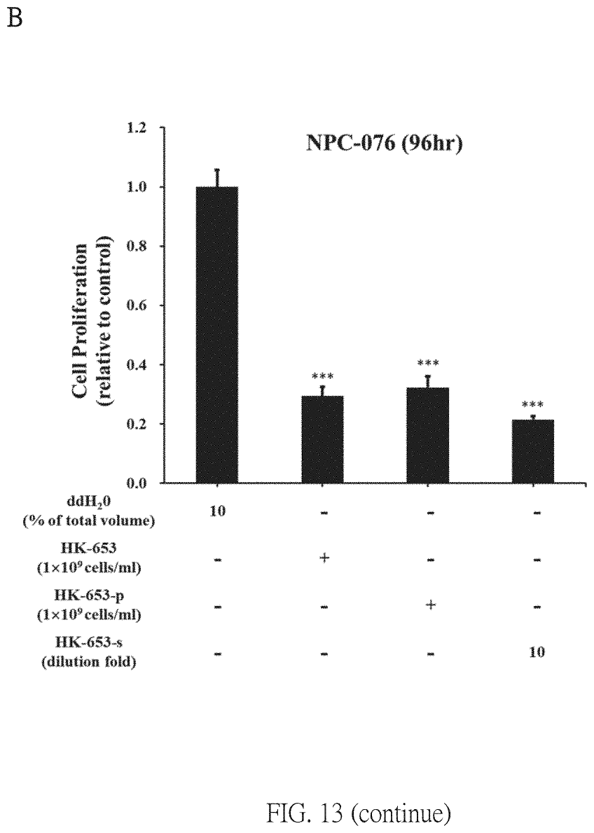

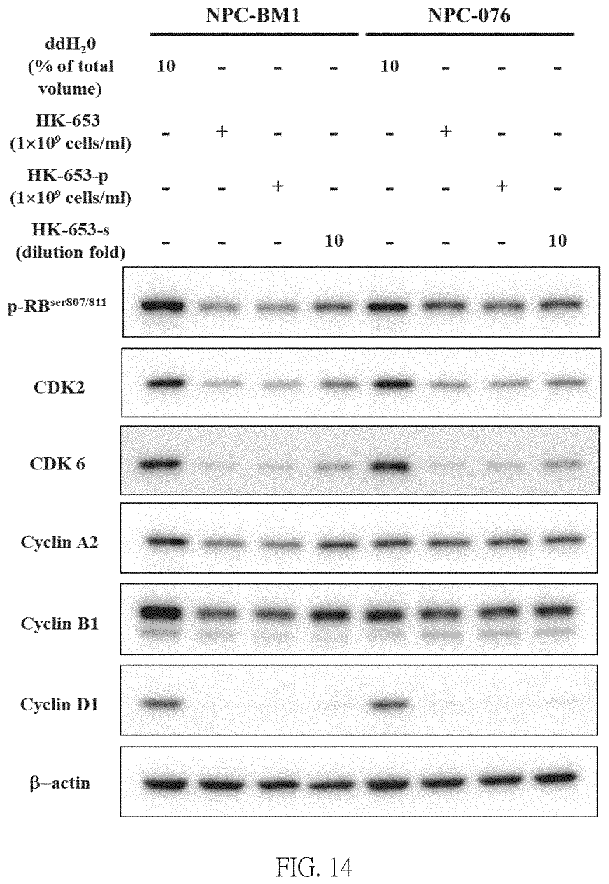

In Embodiment 13, the active substances in heat-killed whole-bacterial liquids of Lactobacillus paracasei GMNL-653 to inhibit proliferations of nasopharyngeal carcinoma cells are analyzed. By high-speed centrifugation (12000 g; 10 minutes), heat-killed whole-bacterial liquids of Lactobacillus paracasei GMNL-653 are separated into heat-killed microbial supernatants (HK-653-s) and heat-killed bacterial pellets (HK-653-p), and their inhibitory effect on nasopharyngeal carcinoma cell proliferations is further analyzed. Referring to FIG. 13, which illustrates the effect of heat-killed whole-bacterial liquids (HK-653), heat-killed microbial supernatants (HK-653-s) or heat-killed bacterial pellets (HK-653-p) of Lactobacillus paracasei GMNL-653 on proliferations of nasopharyngeal carcinoma cells. Nasopharyngeal carcinoma cells, NPC-BM1, (A of FIG. 13) and nasopharyngeal carcinoma cells, NPC-076, (B of FIG. 13) are treated with HK-653, HK-653-s or HK-653-p for 96 hours, and afterwards proliferations of carcinoma cells are determined with the MTT assay. In Embodiment 13, “ddH₂O”, “HK-653”, “HK-653-p” and “HK-653-s” mean “control group” based on double-distilled water (10% of the total treatment volume) for treatment, heat-killed whole-bacterial liquids (1×10⁹ cells/ml) of Lactobacillus paracasei GMNL-653, heat-killed bacterial pellets (1×10⁹ cells/ml) centrifuged from heat-killed whole-bacterial liquids of Lactobacillus paracasei GMNL-653 and re-dissolved in sterile water, and heat-killed microbial supernatants (10-fold dilution) extracted from heat-killed whole-bacterial liquids of Lactobacillus paracasei GMNL-653 from which bacterial pellets are removed, respectively. In statistic analysis, the status for proliferations of carcinoma cells is expressed as the relative proliferation ratio for each treatment calculated relative to the optical density of carcinoma cells treated with “ddH₂O” (“control group”) (“***”: p<0.001). As shown in test results, proliferations of two nasopharyngeal carcinoma cell lines are inhibited by heat-killed microbial supernatants (HK-653-s; 10-fold dilution which is equivalent to whole-bacterial liquids with a concentration of 1×10⁹ cells/ml) or heat-killed bacterial pellets (HK-653-p; 1×10⁹ cells/ml). Referring to FIG. 14, which illustrates the effect of heat-killed whole-bacterial liquids (HK-653), heat-killed microbial supernatants (HK-653-s) or heat-killed bacterial pellets (HK-653-p) of Lactobacillus paracasei GMNL-653 on proteins related to the cell cycle of nasopharyngeal carcinoma cells. The two nasopharyngeal carcinoma cell lines are treated with “ddH₂O”, “HK-653”, “HK-653-p” and “HK-653-s” for 48 hours, and afterwards the expressions of proteins are analyzed with Western blotting in which β-actin is used as a loading control to normalize the levels of protein. As shown in test results, the expressions of proteins related to the cell cycle (p-RB^{ser807/811}, CDK2, CDK6, Cyclin A2, Cyclin B1 and Cyclin D1) are inhibited by heat-killed microbial supernatants (HK-653-s; 10-fold dilution which is equivalent to whole-bacterial liquids with a concentration of 1×10⁹ cells/ml) or heat-killed bacterial pellets (HK-653-p; 1×10⁹ cells/ml).

### Embodiment 14

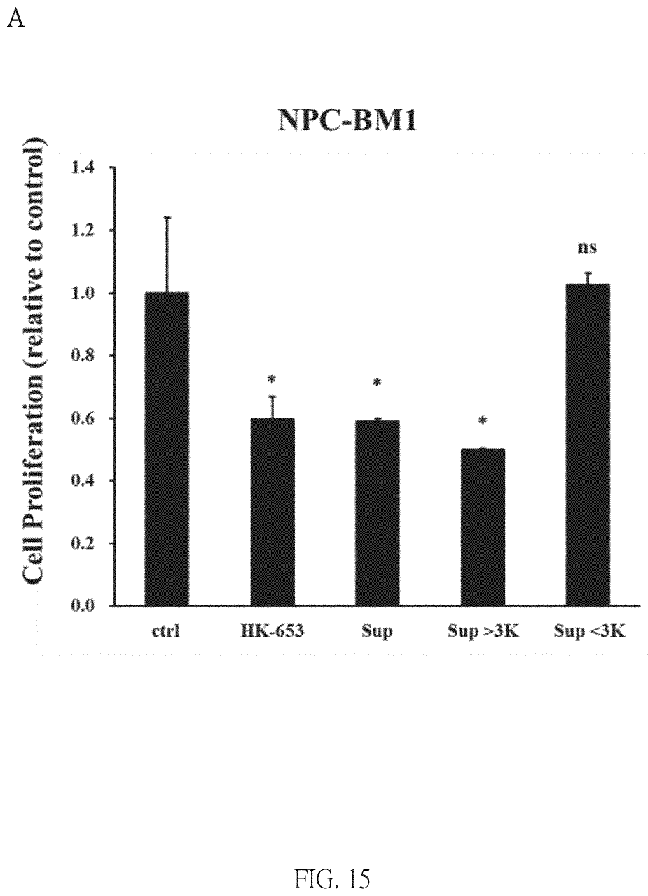

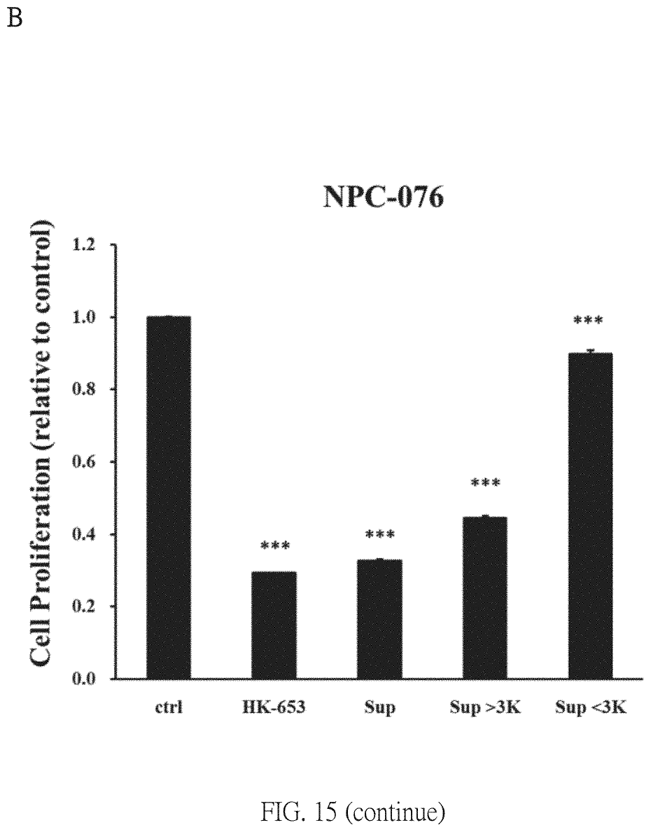

Based on the molecular weight of 3 kDa, heat-killed microbial supernatants (HK-653-s), through an Amicon ultra centrifugal filter, are further divided into two portions, each of which is equivalently diluted, for treating nasopharyngeal carcinoma cells. Referring to FIG. 15, which illustrates the effect of two portions of heat-killed microbial supernatants of Lactobacillus paracasei GMNL-653 containing molecules with the molecular weight greater than 3 kDa (“sup>3K”) or with the molecular weight less than 3 kDa (“sup<3K”) on proliferations of nasopharyngeal carcinoma cells. Nasopharyngeal carcinoma cells, NPC-BM1, (A of FIG. 15) and nasopharyngeal carcinoma cells, NPC-076, (B of FIG. 15) are treated for 72 hours, and afterwards proliferations of carcinoma cells are evaluated with the MTT assay. In FIG. 15, “ctrl”, “HK-653”, “Sup”, “Sup>3K” and “Sup<3K” mean “control group” based on double-distilled water (10% of the total treatment volume) for treatment, heat-killed whole-bacterial liquids (1×10⁹ cells/ml) of Lactobacillus paracasei GMNL-653, heat-killed microbial supernatants (10% of the total treatment volume) extracted from heat-killed whole-bacterial liquids of Lactobacillus paracasei GMNL-653 from which microbial pellets are removed, and two portions of heat-killed microbial supernatants (10% of the total treatment volume) containing molecules with the molecular weight greater than 3 kDa or with the molecular weight less than 3 kDa, respectively. In statistic analysis, the status for proliferations of carcinoma cells is expressed as the relative proliferation ratio for each treatment calculated relative to the optical density of carcinoma cells treated with “ddH₂O” (“control group”) (*: p<0.05″; “**: p<0.01”; “***”: p<0.001; “ns”: no significant difference). As shown in test results, proliferations of nasopharyngeal carcinoma cells are inhibited by the portion of heat-killed microbial supernatants containing molecules with the molecular weight greater than 3 kDa, but not by the portion of heat-killed microbial supernatants containing molecules with the molecular weight less than 3 kDa.

### Embodiment 15

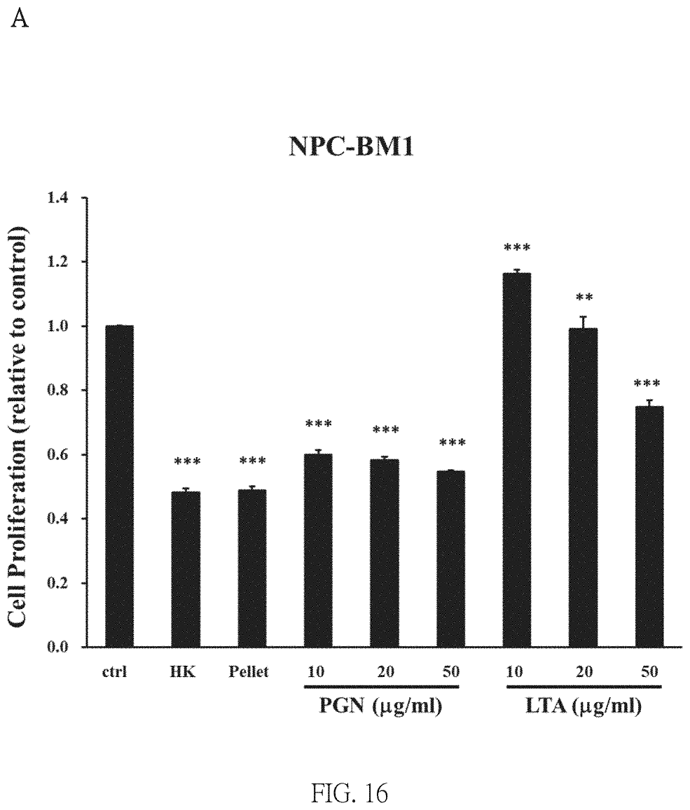

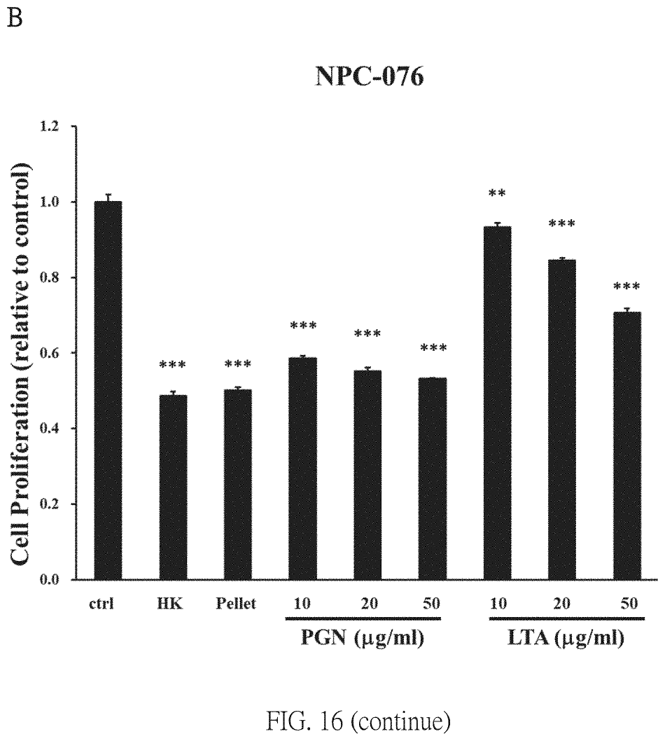

In Embodiment 15, the two nasopharyngeal carcinoma cell lines are further treated with purified cell wall ingredients, lipoteichoic acid (LTA) or peptidoglycan (PGN). Referring to FIG. 16, which illustrates the effect of lipoteichoic acid or peptidoglycan in heat-killed bacterial pellets of Lactobacillus paracasei GMNL-653 on proliferations of nasopharyngeal carcinoma cells. Nasopharyngeal carcinoma cells, NPC-BM1, (A of FIG. 16) and nasopharyngeal carcinoma cells, NPC-076, (B of FIG. 16) are treated for 72 hours, and afterwards proliferations of carcinoma cells are determined with the MTT assay. In FIG. 16, “ctrl”, “HK”, “Pellet”, “LTA” and “PGN” mean “control group” based on double-distilled water for treatment, heat-killed whole-bacterial liquids (1×10⁹ cells/m) of Lactobacillus paracasei GMNL-653, heat-killed bacterial pellets (1×10⁹ cells/ml) centrifuged from heat-killed whole-bacterial liquids of Lactobacillus paracasei GMNL-653 and re-dissolved in sterile water, lipoteichoic acid (10 μg/ml; 20 μg/ml; 50 μg/ml), and peptidoglycan (10 μg/ml; 20 μg/ml; 50 μg/ml), respectively. In statistic analysis, the status for proliferations of carcinoma cells is expressed as the relative proliferation ratio for each treatment calculated relative to the optical density of carcinoma cells treated with double-distilled water (“control group”) (“**”: p<0.01; “***”: p<0.001; “ns”: no significant difference). It can be seen from test results that (1) proliferations of nasopharyngeal carcinoma cells are still inhibited by heat-killed bacterial pellets centrifuged from heat-killed whole-bacterial liquids of Lactobacillus paracasei GMNL-653, (2) proliferations of nasopharyngeal carcinoma cells are inhibited by PGN or LTA in a dose-dependent manner, and (3) proliferations of nasopharyngeal carcinoma cells are more strongly inhibited by PGN than by LTA. Accordingly, it is concluded that the activity for proliferations of nasopharyngeal carcinoma cells is significantly inhibited by PGN or LTA in Lactobacillus paracasei GMNL-653.

In summary, the ingredients in Lactobacillus paracasei GMNL-653 to effectively inhibit nasopharyngeal carcinoma are the portion of heat-killed microbial supernatants containing molecules with the molecular weight greater than 3 kDa, heat-killed bacterial pellets, and lipoteichoic acid as well as peptidoglycan of Lactobacillus paracasei GMNL-653 wherein peptidoglycan of Lactobacillus paracasei GMNL-653 is the ingredient to inhibit nasopharyngeal carcinoma most effectively.

As disclosed in above embodiments, Lactobacillus paracasei or heat-killed whole-bacterial liquids thereof are effective in inhibiting nasopharyngeal carcinoma through pyroptosis or cell cycle arrest. Alternatively, proliferations of nasopharyngeal carcinoma cells or nasopharyngeal carcinoma development is inhibited by increasing production and accumulation of ROS in nasopharyngeal carcinoma cells or by diminishing self-renewals of nasopharyngeal carcinoma stein cells instead of apoptosis.

The embodiments disclosed hereinbefore are aimed to describe technical ideas and features in the present disclosure and make persons skilled in the art comprehend and embody the present invention but not taken as evidences to restrict claims hereinafter, that is, any equivalent change or modification depending on spirit of the present disclosure are still incorporated into the claims.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A composition, comprising: Lactobacillus paracasei GMNL-653 (deposited under the number of BCRC 910721 or CCTCC M 2016226) or heat-killed whole-bacterial liquids thereof as effective ingredients to inhibit proliferations of nasopharyngeal carcinoma cells or reduce a probability of nasopharyngeal carcinoma development through pyroptosis or cell cycle arrest.

2. The composition as claimed in claim 1 wherein proliferations of nasopharyngeal carcinoma cells inhibited or a probability of nasopharyngeal carcinoma development reduced is not induced by the effective ingredients through apoptosis.

3. The composition as claimed in claim 1 wherein the effective ingredients comprise Lactobacillus paracasei GMNL-653 or peptidoglycan and lipoteichoic acid in heat-killed whole-bacterial liquids of Lactobacillus paracasei GMNL-653.

4. The composition as claimed in claim 1 wherein the effective ingredients comprise the portion of separated heat-killed microbial supernates containing molecules with the molecular weight greater than 3 kDa in Lactobacillus paracasei GMNL-653.

5. The composition as claimed in claim 1 wherein the composition is manufactured as a pharmaceutical composition, nutritional supplements or health food.

6. The composition as claimed in claim 5 wherein the composition further comprises a pharmaceutically accepted vehicle.

7. The composition as claimed in claim 5 wherein the composition is manufactured as a solution, a suspension liquid, an emulsion, powders, a pastille, a pill, syrup, an oral ingot, a tablet, a chewing gum, a stiff paste or a capsule.

8. The composition as claimed in claim 5 wherein the composition further comprises an edible material including, without limitation, water, liquid dairy product, milk, concentrated milk, yogurt, frozen yogurt, Lactobacillus fermented beverage, powdered milk, ice cream, cheese, dry cheese, soymilk, fermented soymilk, vegetable juice, fruit juice, sports drink, dessert, jelly, candy, baby food, health food, animal food, Chinese medicinal herb or dietary supplement.

9. A method to treat nasopharyngeal carcinoma with Lactobacillus paracasei GMNL-653 wherein the method comprises administration of Lactobacillus paracasei GMNL-653 which is deposited at China Center for Type Culture Collection (CCTCC) under the number CCTCC M 2016226, or heat-killed whole-bacterial liquids thereof as effective ingredients to an individual for inhibiting proliferations of nasopharyngeal carcinoma or reducing a probability of nasopharyngeal carcinoma development through pyroptosis or cell cycle arrest;
wherein the heat-killed whole-bacterial liquids is a bacterial culture centrifuged to remove supernatant, followed by re-dissolved in water and then heat to kill the bacterium;
wherein the effective ingredients comprise a portion of the heat-killed whole-bacterial liquids of Lactobacillus paracasei GMNL-653, wherein the portion contains molecules with a molecular weight greater than 3 kDa.

10. The method as claimed in claim 9 wherein the inhibited proliferations of nasopharyngeal carcinoma cells or the reduced probability of nasopharyngeal carcinoma development is not induced by the effective ingredients through apoptosis.

11. The method as claimed in claim 9 wherein the effective ingredients comprise Lactobacillus paracasei GMNL-653 or peptidoglycan and lipoteichoic acid in heat-killed whole-bacterial liquids of Lactobacillus paracasei GMNL-653.

12. (canceled)

13. The method as claimed in claim 9 wherein proliferations of nasopharyngeal carcinoma cells are inhibited by the effective ingredients through generation and accumulation of reactive oxygen species in nasopharyngeal carcinoma cells.

14. The method as claimed in claim 9 wherein the probability of nasopharyngeal carcinoma development is reduced by the effective ingredients through inhibition of self-renewals of nasopharyngeal carcinoma stem cells.

15. The method as claimed in claim 11 wherein the effective dosage of peptidoglycan or lipoteichoic acid in Lactobacillus paracasei GMNL-653 or heat-killed whole-bacterial liquids thereof ranges from 10 μg/ml to 50 μg/ml.

16. The method as claimed in claim 9 wherein the effective ingredients are administered to a patient by oral administration, or by injection.
